SEQUENCE LISTING (104 sequences)

SEQ ID NO: 1 - DNA, 3060 bp, Homo sapiens
ccgacctgaagagaaaccgcagctcattagccaaatgcatgagcctcaggcgcgctggaggtgagactaacctctagtcccccgtcgaagccagagagcagtaagagggagcgcccgccgttgatgccccagctgctctggccgcgatgggcactgcaggggctttcctgtgcgcggggtctccagcatctccacgaaggcagagttgggggtctggcagcgcgttctggactttgcccgccgccagtgcgattctccctcccggttccagtcgccgcggacgatgcttcctcccacccaccgcccgcgggctcagagagcaggtccccgcaccgcgcgggctgtgcgcgctccgggcaacatggtccagtgccactacggtttgggcgctgctccaggagctcctgagtctagatctaaccccacttggtccccatggacttgccagaggacttcacactatccacgcctcaagaagcccttctcccgtgaagaccaagttcaggaaatctgaaagcctgacacttgggaactgtcccatagtagctcccagctttgcgtgcccctacctcgcgctccttggaacggccaccaagacgtgaaattttggaagaagatactccgactttagtggaaagacctaaaggaaacgaacagcggcctctgcttctttgagcttggaagagccgctactcgaatgagctcaggcttcctgtggcaaagagagcgaagcggctgtgctcagcccacgccccggcgctgttcctgcccagccaatcagcctcaccacagatgactgctcccggcccggattgactgaatgctgattcctcgagaaactgcgaaacaggttgaatttccaggaggaatgttctggcttccgttgcacctctctagaaagggcaagtagagaaacgcgcatcagctgaatcattggggacatttcaacttatgtagacacgtctttcaaagttcacataagtcttcatatccatataactacaggacgtagttaagggaaattttctcgggattcgcatttaatacagttaatacaaaaactccgacctctccaattctgtatcacctttctgcttcctaacacctttgaaaaggctaggagaaatttttctgcagtggtctttcttcagcatgcttgacagtttctgagttacatctgttcatcctttatttgatgataaatggaatgaaagaaaaaggaagttttaaaaaatatatagagacaggtatttcaaggtacaaatgctatttcaaagtgtgtaatattttaaaacttcagacgtcagatcaatgcccgtgtttttccagggcattttaaattcttacattttaattcaaattagtaaaataaacatagcatgttttagcatattacattagttgtaatttccttccatatttactgccaacatttaatattttttcctgtccactatttacttcaaactgaggaaaagtacgtgcaatctgcacaattcaaaattgtaaagcaatgctaactcacatcagagcttttcttacttcttatgaacatagaggaaataggtttgttgtatcctctagtctacattctatgactttcacatattacctatcagtttattgcataagtttagaagaagaaaatcactttgttttttactgcatagtctgaggatgtttccactttctttaaataactcatacttcaaagccataatttttttctattcttgactacaaattttccttatcttgatatattttgttcttggccttacaatacaatgaagttttcttttgtttttctttcttttttcttttttgagacggagtcttgctttgtcgcccaggctggagtgcagtggcaggatctcggctcactgcaagctccgcctcccgggttcacaccattctcctgcctcagcctcccaggtagctgggactacaggctcccgccaccacgcccggctaattttttgtatttttcgtagagactgggtttcactgtgttagccaggatggtcttgatctcctgacctcgtgatccgcccgcctcggcctcccaaagtgcagggattacaggcgtgagctacagctcggcctctttttttttttttttttttttttttcacaaattgttaattaggcttctaacaggccactattttttccacatgttctaattgcatatgcaagtgtacagcattgttcactgtttaaaggcttaacaacaaccctgtggtccatctggggtaaatgtaaaagctttcatcttcattacatgcgtttttaatcttatttgtgcctatgatctctgttttcacttttgttttgctttgttgctttatctcacctttcatttatgtaatcttgttgtattttatgtatccttttgaatggccttaaaccctccctaaacagtgcaggatataaatacagtgctatactgttatgttgattatactatttctacaatcttgagcatcaggtaaagatctctatcaaagatataagtaagttaaagatcagaataacacagattttaaaaataagattctaccattctattatattgtattccatttctttgctccactgacatgaaagtgtgaatataaaacatgatggacctaaaacaaactagctcttgctactttcaacagtccttggtttcttgaaagacaccagtcttgcatttgtgccaccaccacttctgtcaagctgaacttggctgttctccttaagcttccttaatttctgatgaaacctacctggtccccttgcaagttgacactcatataccttcattcaagcggtgatatttccatttgaagtgactgccacaatctgcaccttcttgtcctccgctctcatgtagccatttcaccaacttgtctatactctgacaacattaaagatttgcctctctgctgcagtctgttctatatgtatctatgggagtaataatatataatgataataataacaactaacatttattaactactggtcatttatcaggaactgccaagcacttcaaagaaattaagtcattta

SEQ ID NO: 2 - DNA, 20 bp, Artificial Sequence
  Description of Artificial Sequence Synthetic primer
tatatgtctcagcctgggcg

SEQ ID NO: 3 - DNA, 20 bp, Artificial Sequence
  Description of Artificial Sequence Synthetic primer
tcacaggagagcagacacgt

SEQ ID NO: 4 - DNA, 20 bp, Artificial Sequence
  Description of Artificial Sequence Synthetic primer
ctcttgctcactctagggac

SEQ ID NO: 5 - DNA, 20 bp, Artificial Sequence
  Description of Artificial Sequence Synthetic primer
caaatatgatcatgagccac

SEQ ID NO: 6 - DNA, 22 bp, Artificial Sequence
  Description of Artificial Sequence Synthetic primer
cacatatcatctgagaagccca

SEQ ID NO: 7 - DNA, 20 bp, Artificial Sequence
  Description of Artificial Sequence Synthetic primer
aggacacaccacttcactgc

SEQ ID NO: 8 - DNA, 20 bp, Artificial Sequence
  Description of Artificial Sequence Synthetic primer
aggcagtgaagtggtgtgtc

SEQ ID NO: 9 - DNA, 20 bp, Artificial Sequence
  Description of Artificial Sequence Synthetic primer
accttcattcaagcggtgat

SEQ ID NO: 10 - DNA, 20 bp, Artificial Sequence
  Description of Artificial Sequence Synthetic primer
atgagagcggaggacaagaa

SEQ ID NO: 11 - DNA, 20 bp, Artificial Sequence
  Description of Artificial Sequence Synthetic primer
aaccctccctaaacagtgca

SEQ ID NO: 12 - DNA, 21 bp, Artificial Sequence
  Description of Artificial Sequence Synthetic primer
gagatctttacctgatgctca

SEQ ID NO: 13 - DNA, 20 bp, Artificial Sequence
  Description of Artificial Sequence Synthetic primer
aggcttctaacaggccacta

SEQ ID NO: 14 - DNA, 20 bp, Artificial Sequence
  Description of Artificial Sequence Synthetic primer
aacaatgctgtacacttgca

SEQ ID NO: 15 - DNA, 20 bp, Artificial Sequence
  Description of Artificial Sequence Synthetic primer
cttggccttacaatacaatg

SEQ ID NO: 16 - DNA, 20 bp, Artificial Sequence
  Description of Artificial Sequence Synthetic primer
cgacaaagcaagactccgtc

SEQ ID NO: 17 - DNA, 22 bp, Artificial Sequence
  Description of Artificial Sequence Synthetic primer
ccttccatatttactgccaaca

SEQ ID NO: 18 - DNA, 19 bp, Artificial Sequence
  Description of Artificial Sequence Synthetic primer
gaattgtgcagattgcacg

SEQ ID NO: 19 - DNA, 20 bp, Artificial Sequence
  Description of Artificial Sequence Synthetic primer
tccgacctctccaattctgt

SEQ ID NO: 20 - DNA, 20 bp, Artificial Sequence
  Description of Artificial Sequence Synthetic primer
agcatgctgaagaaagacca

SEQ ID NO: 21 - DNA, 20 bp, Artificial Sequence
  Description of Artificial Sequence Synthetic primer
tcagcctcaccacagatgac

SEQ ID NO: 22 - DNA, 20 bp, Artificial Sequence
  Description of Artificial Sequence Synthetic primer
ctcgaggaatcagcattcag

SEQ ID NO: 23 - DNA, 20 bp, Artificial Sequence
  Description of Artificial Sequence Synthetic primer
gtccagtgccactacggttt

SEQ ID NO: 24 - DNA, 20 bp, Artificial Sequence
  Description of Artificial Sequence Synthetic primer
gggaccaagtggggttagat

SEQ ID NO: 25 - DNA, 20 bp, Artificial Sequence
  Description of Artificial Sequence Synthetic primer
cttctttgctcctccattgc

SEQ ID NO: 26 - DNA, 30 bp, Artificial Sequence
  Description of Artificial Sequence Synthetic primer
aaagctagctgtcagtggagcaaagaaatg

SEQ ID NO: 27 - DNA, 25 bp, Artificial Sequence
  Description of Artificial Sequence Synthetic primer
aaagctagcctcgcgctccttggaa

SEQ ID NO: 28 - DNA, 26 bp, Artificial Sequence
  Description of Artificial Sequence Synthetic oligonucleotide
aaatgaaaggtgagataaagcaacaa

SEQ ID NO: 29 - DNA, 26 bp, Artificial Sequence
  Description of Artificial Sequence Synthetic oligonucleotide
aaatgaaaggtgaaataaagcaacaa

SEQ ID NO: 30 - DNA, 26 bp, Artificial Sequence
  Description of Artificial Sequence Synthetic oligonucleotide
gagctcattcgagtagcggctcttcc

SEQ ID NO: 31 - DNA, 26 bp, Artificial Sequence
  Description of Artificial Sequence Synthetic oligonucleotide
gagctcattcgagcagcggctcttcc

SEQ ID NO: 32 - DNA, 9 bp, Artificial Sequence
  Description of Artificial Sequence Synthetic oligonucleotide
agagtatag

SEQ ID NO: 33 - DNA, 9 bp, Artificial Sequence
  Description of Artificial Sequence Synthetic oligonucleotide
agagcatag

SEQ ID NO: 34 - DNA, 9 bp, Artificial Sequence
  Description of Artificial Sequence Synthetic oligonucleotide
agggtttaa

SEQ ID NO: 35 - DNA, 9 bp, Artificial Sequence
  Description of Artificial Sequence Synthetic oligonucleotide
agggcttaa

SEQ ID NO: 36 - DNA, 9 bp, Artificial Sequence
  Description of Artificial Sequence Synthetic oligonucleotide
gtgagataa

SEQ ID NO: 37 - DNA, 9 bp, Artificial Sequence
  Description of Artificial Sequence Synthetic oligonucleotide
gtgaaataa

SEQ ID NO: 38 - DNA, 9 bp, Artificial Sequence
  Description of Artificial Sequence Synthetic oligonucleotide
atggtgtga

SEQ ID NO: 39 - DNA, 9 bp, Artificial Sequence
  Description of Artificial Sequence Synthetic oligonucleotide
atggcgtga

SEQ ID NO: 40 - DNA, 9 bp, Artificial Sequence
  Description of Artificial Sequence Synthetic oligonucleotide
attatggct

SEQ ID NO: 41 - DNA, 9 bp, Artificial Sequence
  Description of Artificial Sequence Synthetic oligonucleotide
attacggct

SEQ ID NO: 42 - DNA, 9 bp, Artificial Sequence
  Description of Artificial Sequence Synthetic oligonucleotide
ctggaaaaa

SEQ ID NO: 43 - DNA, 9 bp, Artificial Sequence
  Description of Artificial Sequence Synthetic oligonucleotide
ctgggaaaa

SEQ ID NO: 44 - DNA, 9 bp, Artificial Sequence
  Description of Artificial Sequence Synthetic oligonucleotide
cccaatgat

SEQ ID NO: 45 - DNA, 9 bp, Artificial Sequence
  Description of Artificial Sequence Synthetic oligonucleotide
cccagtgat

SEQ ID NO: 46 - DNA, 9 bp, Artificial Sequence
  Description of Artificial Sequence Synthetic oligonucleotide
cgagtagcg

SEQ ID NO: 47 - DNA, 9 bp, Artificial Sequence
  Description of Artificial Sequence Synthetic oligonucleotide
cgagcagcg

SEQ ID NO: 48 - DNA, 9 bp, Artificial Sequence
  Description of Artificial Sequence Synthetic oligonucleotide
agggcctga

SEQ ID NO: 49 - DNA, 9 bp, Artificial Sequence
  Description of Artificial Sequence Synthetic oligonucleotide
agggtctga

SEQ ID NO: 50 - DNA, 9 bp, Artificial Sequence
  Description of Artificial Sequence Synthetic oligonucleotide
aggtgctgg

SEQ ID NO: 51 - DNA, 9 bp, Artificial Sequence
  Description of Artificial Sequence Synthetic oligonucleotide
aggttctgg

SEQ ID NO: 52 - DNA, 9 bp, Artificial Sequence
  Description of Artificial Sequence Synthetic oligonucleotide
aggtactgg

SEQ ID NO: 53 - DNA, 9 bp, Artificial Sequence
  Description of Artificial Sequence Synthetic oligonucleotide
agatcgtga

SEQ ID NO: 54 - DNA, 9 bp, Artificial Sequence
  Description of Artificial Sequence Synthetic oligonucleotide
agattgtga

SEQ ID NO: 55 - DNA, 9 bp, Artificial Sequence
  Description of Artificial Sequence Synthetic oligonucleotide
aatggtatg

SEQ ID NO: 56 - DNA, 9 bp, Artificial Sequence
  Description of Artificial Sequence Synthetic oligonucleotide
aatgttatg

SEQ ID NO: 57 - DNA, 9 bp, Artificial Sequence
  Description of Artificial Sequence Synthetic oligonucleotide
gcaacaatg

SEQ ID NO: 58 - DNA, 9 bp, Artificial Sequence
  Description of Artificial Sequence Synthetic oligonucleotide
gcaataatg

SEQ ID NO: 59 - DNA, 9 bp, Artificial Sequence
  Description of Artificial Sequence Synthetic oligonucleotide
ttactaatt

SEQ ID NO: 60 - DNA, 9 bp, Artificial Sequence
  Description of Artificial Sequence Synthetic oligonucleotide
ttacaaatt

SEQ ID NO: 61 - DNA, 16 bp, Artificial Sequence
  Description of Artificial Sequence Synthetic probe
aggtgagataaagcaa

SEQ ID NO: 62 - DNA, 16 bp, Artificial Sequence
  Description of Artificial Sequence Synthetic probe
tgaaaggtgaaataaa

SEQ ID NO: 63 - DNA, 26 bp, Artificial Sequence
  Description of Artificial Sequence Synthetic primer
aaggccattcaaaaggatacataaaa

SEQ ID NO: 64 - DNA, 26 bp, Artificial Sequence
  Description of Artificial Sequence Synthetic primer
tctgttttcacttttgttttgctttg

SEQ ID NO: 65 - DNA, 15 bp, Artificial Sequence
  Description of Artificial Sequence Synthetic probe
tccccaatgattcag

SEQ ID NO: 66 - DNA, 14 bp, Artificial Sequence
  Description of Artificial Sequence Synthetic probe
ccccagtgattcag

SEQ ID NO: 67 - DNA, 26 bp, Artificial Sequence
  Description of Artificial Sequence Synthetic primer
tgtgaactttgaaagacgtgtctaca

SEQ ID NO: 68 - DNA, 22 bp, Artificial Sequence
  Description of Artificial Sequence Synthetic primer
caagtagagaaacgcgcatcag

SEQ ID NO: 69 - DNA, 15 bp, Artificial Sequence
  Description of Artificial Sequence Synthetic probe
ttcgagtagcggctc

SEQ ID NO: 70 - DNA, 13 bp, Artificial Sequence
  Description of Artificial Sequence Synthetic probe
tcgagcagcggct

SEQ ID NO: 71 - DNA, 18 bp, Artificial Sequence
  Description of Artificial Sequence Synthetic primer
ccgcttcgctctctttgc

SEQ ID NO: 72 - DNA, 22 bp, Artificial Sequence
  Description of Artificial Sequence Synthetic primer
cctctgcttctttgagcttgga

SEQ ID NO: 73 - DNA, 14 bp, Artificial Sequence
  Description of Artificial Sequence Synthetic probe
ctcacgatctcttc

SEQ ID NO: 74 - DNA, 14 bp, Artificial Sequence
  Description of Artificial Sequence Synthetic probe
cctcacaatctctt

SEQ ID NO: 75 - DNA, 18 bp, Artificial Sequence
  Description of Artificial Sequence Synthetic primer
aacagccgggtggtgtca

SEQ ID NO: 76 - DNA, 22 bp, Artificial Sequence
  Description of Artificial Sequence Synthetic primer
atgtatgttggcctcctttgct

SEQ ID NO: 77 - DNA, 16 bp, Artificial Sequence
  Description of Artificial Sequence Synthetic probe
ctggttgtagggcttt

SEQ ID NO: 78 - DNA, 17 bp, Artificial Sequence
  Description of Artificial Sequence Synthetic probe
cctggttatagggcttt

SEQ ID NO: 79 - DNA, 32 bp, Artificial Sequence
  Description of Artificial Sequence Synthetic primer
cgggcttcattcagaattttttatctttgatt

SEQ ID NO: 80 - DNA, 25 bp, Artificial Sequence
  Description of Artificial Sequence Synthetic primer
caccaagtagaacaaatgccaaaca

SEQ ID NO: 81 - DNA, 16 bp, Artificial Sequence
  Description of Artificial Sequence Synthetic probe
atgctaccgatgtcac

SEQ ID NO: 82 - DNA, 16 bp, Artificial Sequence
  Description of Artificial Sequence Synthetic probe
atgctaccaatgtcac

SEQ ID NO: 83 - DNA, 21 bp, Artificial Sequence
  Description of Artificial Sequence Synthetic primer
tggtcctcagagggatcactt

SEQ ID NO: 84 - DNA, 23 bp, Artificial Sequence
  Description of Artificial Sequence Synthetic primer
tccttcactccacctaccttctc

SEQ ID NO: 85 - DNA, 17 bp, Artificial Sequence
  Description of Artificial Sequence Synthetic probe
aagtaaggtctctttcc

SEQ ID NO: 86 - DNA, 17 bp, Artificial Sequence
  Description of Artificial Sequence Synthetic probe
aagtaaggtctttttcc

SEQ ID NO: 87 - DNA, 25 bp, Artificial Sequence
  Description of Artificial Sequence Synthetic primer
gcttgctgaggaaaagttggacata

SEQ ID NO: 88 - DNA, 33 bp, Artificial Sequence
  Description of Artificial Sequence Synthetic primer
agttgcaggaaatcaaagataaaaaattctgaa

SEQ ID NO: 89 - DNA, 16 bp, Artificial Sequence
  Description of Artificial Sequence Synthetic probe
catccactgcagacag

SEQ ID NO: 90 - DNA, 14 bp, Artificial Sequence
  Description of Artificial Sequence Synthetic probe
tccactgcaaacag

SEQ ID NO: 91 - DNA, 26 bp, Artificial Sequence
  Description of Artificial Sequence Synthetic primer
gccagagctacctaccaaaatttaga

SEQ ID NO: 92 - DNA, 20 bp, Artificial Sequence
  Description of Artificial Sequence Synthetic primer
gcctttcaacaggccattgg

SEQ ID NO: 93 - DNA, 15 bp, Artificial Sequence
  Description of Artificial Sequence Synthetic probe
aggccaaggcagaag

SEQ ID NO: 94 - DNA, 15 bp, Artificial Sequence
  Description of Artificial Sequence Synthetic probe
aggccaagacagaag

SEQ ID NO: 95 - DNA, 18 bp, Artificial Sequence
  Description of Artificial Sequence Synthetic primer
gcaatcccagccctttgg

SEQ ID NO: 96 - DNA, 24 bp, Artificial Sequence
  Description of Artificial Sequence Synthetic primer
ctcaaactccaggcttcaacaatc

SEQ ID NO: 97 - DNA, 19 bp, Artificial Sequence
  Description of Artificial Sequence Synthetic probe
ctgtttctccaacggtgta

SEQ ID NO: 98 - DNA, 20 bp, Artificial Sequence
  Description of Artificial Sequence Synthetic probe
actgtttctccaatggtgta

SEQ ID NO: 99 - DNA, 19 bp, Artificial Sequence
  Description of Artificial Sequence Synthetic primer
ccaacttggccaggaagga

SEQ ID NO: 100 - DNA, 21 bp, Artificial Sequence
  Description of Artificial Sequence Synthetic primer
ggcatccacagacatcaggtt

SEQ ID NO: 101 - DNA, 19 bp, Artificial Sequence
  Description of Artificial Sequence Synthetic probe
tcaaaggagaagtggttta

SEQ ID NO: 102 - DNA, 20 bp, Artificial Sequence
  Description of Artificial Sequence Synthetic probe
ttcaaaggagaaatggttta

SEQ ID NO: 103 - DNA, 25 bp, Artificial Sequence
  Description of Artificial Sequence Synthetic primer
ctggatctgagtttctggattctgt

SEQ ID NO: 104 - DNA, 25 bp, Artificial Sequence
  Description of Artificial Sequence Synthetic primer
gggatgttttgcaaactgttctttg

(19) United States
(12) Patent Application Publication
(10) Pub. No.: US 2006/0216738 A1
(43) Pub. Date: Sep. 28, 2006

(54) SNPs in 5' regulatory region of MDR1 gene

(76) Inventors: Morimasa Wada, Kasuga-shi (JP); Michihiko Kuwano, Fukuoka-shi (JP)

Correspondence Address: FROMMER LAWRENCE & HAUG, 745 FIFTH AVENUE- 10TH FL., NEW YORK, NY, 10151, US

(21) Appl. No.: 11/388,647

(22) Filed: Mar. 24, 2006

Related U.S. Application Data
(63) Continuation-in-part of application No. PCT/JP04/13839, filed on Sep. 22, 2004.

(30) Foreign Application Priority Data
Sep. 24, 2003 (JP) 2003-332584
Jun. 18, 2004 (JP) 2004-181914

(51) Int. Cl. C12Q 1/68

(52) U.S. Cl. 435/6

(57) ABSTRACT

The present invention relates to a method for determining haplotypes or diplotypes of a MDR1 gene targeting the 5′ upstream regulatory region of MDR1 gene encoding P-gp, an ABC transporter which is may be expressed in the apical membrane side and may transport a wide range of substrates. By detecting a polymorphism at −934 and/or −692, in addition to a position selected from −2903, −2410, −2352, −1910, −1717, and −1325 in a nucleotide sequence of the 5′ upstream regulatory region of MDR1 gene, haplotypes or diplotypes of the 5′upstream regulatory region of MDR1 gene may be determinable. The above positions to detect polymorphism are indicated in relation to a first base of ATG start codon which is set to +1. ATG start codon is located in exon 2, and the transcription start site corresponds to −699 in this numbering system.

### INCORPORATION BY REFERENCE

This application is a continuation-in-part application of international patent application Serial No. PCT/JP2004/013839 filed Sep. 22, 2004, which claims benefit of Japanese patent application Serial Nos. 2003-332584, filed Sep. 24, 2003, and 2004-181914, filed Jun. 18, 2004.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention relates to a method for determining haplotypes and/or diplotypes of the 5′ regulatory region of MDR1 (multidrug resistance 1) gene, particularly to a method for determining haplotypes and/or diplotypes of the 5′ regulatory region of MDR1 gene, by detecting one or more SNPs (single nucleotide polymorphisms) at positions including −2903, −2410, −2352, −1910, −17170 and −1325, when the position is expressed in relation to a first base of translation start codon ATG which is set to +1, in the nucleotide sequence of the 5′ regulatory region of MDR1 gene, and the like.

### BACKGROUND OF THE INVENTION

Drugs are detoxified and conjugated in vivo and then exported out of the cells. The activity of the detoxification system affects the pharmacokinetics of drugs. Many recent studies have correlated polymorphisms of detoxification-related genes such as cytochrome P450s and glutathione S-transferases with the efficacy and side effects of drugs (see for example, Roden, D. M. and George, A. L., Jr. The genetic basis of variability in drug responses. Nat Rev Drug Discov, 1: 37-44, 2002; Evans, W. E. and Relling, M. V. Pharmacogenomics: translating functional genomics into rational therapeutics. Science, 286: 487-491, 1999; Gonzalez, F. J., Skoda, R. C., Kimura, S., Umeno, M., Zanger, U. M., Nebert, D. W., Gelboin, H. V., Hardwick, J. P., and Meyer, U. A. Characterization of the common genetic defect in humans deficient in debrisoquine metabolism. Nature, 331: 442-446, 1988). On the other hand, the export of drugs has been shown to involve a group of proteins belonging to ATP Binding Cassette (ABC3) transporters (see for example, Konig, J., Nies, A. T., Cui, Y., Leier, I., and Keppler, D. Conjugate export pumps of the multidrug resistance protein (MRP) family: localization, substrate specificity, and MRP2-mediated drug resistance. Biochim Biophys Acta, 1461: 377-394, 1999; Gottesman, M. M., Fojo, T., and Bates, S. E. Multidrug resistance in cancer: role of ATP-dependent transporters. Nat Rev Cancer, 2: 48-58, 2002; Borst, P., Evers, R., Kool, M., and Wijnholds, J. A family of drug transporters: the multidrug resistance-associated proteins. J Natl Cancer Inst, 92: 1295-1302, 2000; Holland, I. B., Cole, S. P. C., Kuchler, K., and Higgins, C. F. ABC proteins, from bacteria to man, p. 423-443. UK: Academic Press, 2003; Wada, M., Uchiumi, T., and Kuwano, M. Canalicular multispecific organic anion transporter, ABCC2. In: S. Broer and C. A. Wagner (eds.), MEMBRANE TRANSPORT DISEASES—Molecular basis of inherited transport defects-, NY: Kluwer Academic/Plenum Publishers, in press, 2003; Kuwano, M., Uchiumi, T., Hayakawa, H., Ono, M., Wada, M., Izumi, H., and Kohno, K. The basic and clinical implications of ABC transporters, Y-box-binding protein-1 (YB-1) and angiogenesis-related factors in human malignancies. Cancer Sci, 94: 9-14, 2003.). A member of ABC transporters, P-glycoprotein (P-gp), affects the pharmacokinetics of drugs by limiting the rate at which they are absorbed. Thus, inter-individual variations in the levels of activity and expression of ABC transporters might be a critical factor in the development of pharmacokinetics.

The MDR1 gene is a known gene that encodes a 170-kDa transmembrane protein, P-gp, located at the cytoplasmic surface of the cell, and its nucleotide sequence is also knwon. P-gp consists of two membrane-spanning domains and two nucleotide-binding domains. Of the various molecular targets, P-gp expression is responsible for cell resistance to the widest variety of anti-cancer drugs (see for example, Scherf, U., Ross, D. T., Waltham, M., Smith, L. H., Lee, J. K., Tanabe, L., Kohn, K. W., Reinhold, W. C., Myers, T. G, Andrews, D. T., Scudiero, D. A., Eisen, M. B., Sausville, E. A., Pommier, Y., Botstein, D., Brown, P. O., and Weinstein, J. N. A gene expression database for the molecular pharmacology of cancer. Nat Genet, 24: 236-244, 2000; Fojo, A. T., Ueda, K., Slamon, D. J., Poplack, D. G, Gottesman, M. M., and Pastan, I. Expression of a multidrug-resistance gene in human tumors and tissues. Proc Natl Acad Sci USA, 84: 265-269, 1987). P-gp overexpression plays an important role in the acquisition of drug resistance in various cancer cells. The enhanced expression of the MDR1 gene in malignant cancer cells has been attributed to various mechanisms, including nuclear translocation of YB-1 (see for example, Bargou, R. C., Jurchott, K., Wagener, C., Bergmann, S., Metzner, S., Bommert, K., Mapara, M. Y., Winzer, K. J., Dietel, M., Dorken, B., and Royer, H. D. Nuclear localization and increased levels of transcription factor YB-1 in primary human breast cancers are associated with intrinsic MDR1 gene expression. Nat Med, 3: 447-450, 1997; Ohga, T., Uchiumi, T., Makino, Y., Koike, K., Wada, M., Kuwano, M., and Kohno, K. Direct involvement of the Y-box binding protein YB-1 in genotoxic stress-induced activation of the human multidrug resistance 1 gene. J Biol Chem, 273: 5997-6000, 1998), promoter rearrangement (see for example, Harada, T., Nagayama, J., Kohno, K., Mickley, L. A., Fojo, T., Kuwano, M., and Wada, M. Alu-associated interstitial deletions and chromosomal re-arrangement in 2 human multidrug-resistant cell lines. Int J Cancer, 86: 506-511, 2000), and alteration of methylation status at CpG sites on the MDR1 promoter (see for example, Kusaba, H., Nakayama, M., Harada, T., Nomoto, M., Kohno, K., Kuwano, M., and Wada, M. Association of 5′ CpG demethylation and altered chromatin structure in the promoter region with transcriptional activation of the multidrug resistance 1 gene in human cancer cells. Eur J Biochem, 262: 924-932, 1999; Nakayama, M., Wada, M., Harada, T., Nagayama, J., Kusaba, H., Ohshima, K., Kozuru, M., Komatsu, H., Ueda, R., and Kuwano, M. Hypomethylation status of CpG sites at the promoter region and overexpression of the human MDR1 gene in acute myeloid leukemias. Blood, 92: 4296-4307, 1998; Tada, Y., Wada, M., Kuroiwa, K., Kinugawa, N., Harada, T., Nagayama, J., Nakagawa, M., Naito, S., and Kuwano, M. MDR1 gene overexpression and altered degree of methylation at the promoter region in bladder cancer during chemotherapeutic treatment. Clin Cancer Res, 6: 4618-4627, 2000).

P-gp is expressed in normal cells of various organs, such as intestine, liver, kidney, brain, and placenta (see for example, Thiebaut, F., Tsuruo, T., Hamada, H., Gottesman, M. M., Pastan, I., and Willingham, M. C. Cellular localization of the multidrug-resistance gene product P-glycoprotein in normal human tissues. Proc Natl Acad Sci USA, 84: 7735-7738, 1987; Sugawara, I., Kataoka, I., Morishita, Y., Hamada, H., Tsuruo, T., Itoyama, S., and Mori, S. Tissue distribution of P-glycoprotein encoded by a multidrug-resistant gene as revealed by a monoclonal antibody, MRK 16. Cancer Res, 48: 1926-1929, 1988; Cordon-Cardo, C., O'Brien, J. P., Casals, D., Rittman-Grauer, L., Biedler, J. L., Melamed, M. R., and Bertino, J. R. Multidrug-resistance gene (P-glycoprotein) is expressed by endothelial cells at blood-brain barrier sites. Proc Natl Acad Sci USA, 86: 695-698, 1989). P-gp's wide substrate specificity and apical localization strongly suggest that it plays a critical role in drug disposition in the human body as well as in animal model (see for example, Schinkel, A. H., Mayer, U., Wagenaar, E., Mol, C. A., van Deemter, L., Smit, J. J., van der Valk, M. A., Voordouw, A. C., Spits, H., van Tellingen, O., Zijlmans, J. M., Fibbe, W. E., and Borst, P. Normal viability and altered pharmacokinetics in mice lacking mdr1-type (drug-transporting) P-glycoproteins. Proc Natl Acad Sci USA, 94: 4028-4033, 1997; Schinkel, A. H. Pharmacological insights from P-glycoprotein knockout mice. Int J Clin Pharmacol Ther, 36: 9-13, 1998; Ambudkar, S. V., Dey, S., Hrycyna, C. A., Ramachandra, M., Pastan, I., and Gottesman, M. M. Biochemical, cellular, and pharmacological aspects of the multidrug transporter. Annu Rev Pharmacol Toxicol, 39: 361-398, 1999; Watkins, P. B. The barrier function of CYP3A4 and P-glycoprotein in the small bowel. Adv Drug Deliv Rev, 27: 161-170, 1997; Fromm, M. F. The influence of MDR1 polymorphisms on P-glycoprotein expression and function in humans. Adv Drug Deliv Rev, 54: 1295-1310, 2002). In the intestine, P-gp is thought to participate in drug absorption after drug ingestion (see for example, Cordon-Cardo, C., O'Brien, J. P., Boccia, J., Casals, D., Bertino, J. R., and Melamed, M. R. Expression of the multidrug resistance gene product (P-glycoprotein) in human normal and tumor tissues. J Histochem Cytochem, 38: 1277-1287, 1990; Schuetz, E. G., Schinkel, A. H., Relling, M. V., and Schuetz, J. D. P-glycoprotein: a major determinant of rifampicin-inducible expression of cytochrome P4503A in mice and humans. Proc Natl Acad Sci USA, 93: 4001-4005, 1996; Greiner, B., Eichelbaum, M., Fritz, P., Kreichgauer, H. P., von Richter, O., Zundler, J., and Kroemer, H. K. The role of intestinal P-glycoprotein in the interaction of digoxin and rifampin. J Clin Invest, 104: 147-153, 1999). At the blood brain barrier, P-gp influences the uptake of substrates into brain (see for example, Thiebaut, F., Tsuruo, T., Hamada, H., Gottesman, M. M., Pastan, I., and Willingham, M. C. Cellular localization of the multidrug-resistance gene product P-glycoprotein in normal human tissues. Proc Natl Acad Sci USA, 84: 7735-7738, 1987; Schumacher, U. and Mollgard, K. The multidrug-resistance P-glycoprotein (Pgp, MDR1) is an early marker of blood-brain barrier development in the microvessels of the developing human brain. Histochem Cell Biol, 108: 179-182, 1997; Rao, V. V., Dahlheimer, J. L., Bardgett, M. E., Snyder, A. Z., Finch, R. A., Sartorelli, A. C., and Piwnica-Worms, D. Choroid plexus epithelial expression of MDR1 P glycoprotein and multidrug resistance-associated protein contribute to the blood-cerebrospinal-fluid drug-permeability barrier. Proc Natl Acad Sci USA, 96: 3900-3905, 1999; Thiebaut, F., Tsuruo, T., Hamada, H., Gottesman, M. M., Pastan, I., and Willingham, M. C. Immunohistochemical localization in normal tissues of different epitopes in the multidrug transport protein P170: evidence for localization in brain capillaries and crossreactivity of one antibody with a muscle protein. J Histochem Cytochem, 37: 159-164, 1989). Since MDR1 expression levels vary widely among individuals (see for example, Hinoshita, E., Uchiumi, T., Taguchi, K., Kinukawa, N., Tsuneyoshi, M., Maehara, Y., Sugimachi, K., and Kuwano, M. Increased expression of an ATP-binding cassette superfamily transporter, multidrug resistance protein 2, in human colorectal carcinomas. Clin Cancer Res, 6: 2401-2407, 2000), these variations may affect the toxicity of drugs and the efficacy of drug treatment from individual to individual through different drug dispositions. Furthermore, these variations may have another clinical relevance as a cancer risk factor, because the present inventors have recently observed the suppression of polyp formation in mdr1a, mouse ortholog of MDR1, -disrupted mice (see for example, Mochida, Y., Taguchi, K., Taniguchi, S., Tsuneyoshi, M., Kuwano, H., Tsuzuki, T., Kuwano, M., and Wada, M. The role of P-glycoprotein in intestinal tumorgenesis: disruption of mdr1a suppresses polyp formation in Apc Min/+mice. Carcinogenesis, 24: 1219-1224, 2003; Yamada, T., Mori, Y., Hayashi, R., Takada, M., Ino, Y., Naishiro, Y., Kondo, T., and Hirohashi, S. Suppression of intestinal polyposis in Mdr1-deficient ApcMin/+mice. Cancer Res, 63: 895-901, 2003). On a molecular basis, however, the mechanism underlying the inter-individual variations in the basal expression level is unknown.

Genetic polymorphisms and their association with P-gp level in MDR1 have been reported recently (see for example, Hoffineyer, S., Burk, O., von Richter, O., Arnold, H. P., Brockmoller, J., Johne, A., Cascorbi, I., Gerloff, T., Roots, I., Eichelbaum, M., and Brinkmann, U. Functional polymorphisms of the human multidrug-resistance gene: multiple sequence variations and correlation of one allele with P-glycoprotein expression and activity in vivo. Proc Natl Acad Sci USA, 97: 3473-3478, 2000; Ito, S., Ieiri, I., Tanabe, M., Suzuki, A., Higuchi, S., and Otsubo, K. Polymorphism of the ABC transporter genes, MDR1, MRP1 and MRP2/cMOAT, in healthy Japanese subjects. Pharmacogenetics, 11: 175-184, 2001). Hoffineyer et al. reported 15 single nucleotide polymorphisms (SNPs), including six in the coding region, in healthy Caucasians. They suggested that one of the SNPs, c.3435C<T (exon 26), is correlated with intestinal P-gp expression and uptake of orally administered digoxin, a P-gp substrate. In Japanese subjects, however, c.3435C>T was reported not to be related to placental expression of P-gp (see for example, Tanabe, M., Ieiri, I., Nagata, N., Inoue, K., Ito, S., Kanamori, Y., Takahashi, M., Kurata, Y., Kigawa, J., Higuchi, S., Terakawa, N., and Otsubo, K. Expression of P-glycoprotein in human placenta: relation to genetic polymorphism of the multidrug resistance (MDR)-1 gene. J Pharmacol Exp Ther, 297: 1137-1143, 2001). Furthermore, in contrast with the report by Hoffineyer et al., a T allele of c.3435C>T increased the expression level of MDR1 mRNA in duodenal enterocytes of healthy Japanese subjects (see for example, Nakamura, T., Sakaeda, T., Horinouchi, M., Tamura, T., Aoyama, N., Shirakawa, T., Matsuo, M., Kasuga, M., and Okumura, K. Effect of the mutation (C3435T) at exon 26 of the MDR1 gene on expression level of MDR1 messenger ribonucleic acid in duodenal enterocytes of healthy Japanese subjects. Clin Pharmacol Ther, 71: 297-303, 2002). c.3435C>T is a silent mutation that does not cause amino acid substitution. Kim et al. (see for example, Kim, R. B., Leake, B. F., Choo, E. F., Dresser, G. K., Kubba, S. V., Schwarz, U. I., Taylor, A., Xie, H. G., McKinsey, J., Zhou, S., Lan, L. B., Schuetz, J. D., Schuetz, E. G., and Wilkinson, G. R. Identification of functionally variant MDR1 alleles among European Americans and African Americans. Clin Pharmacol Ther, 70: 189-199, 2001) reported that P-gp function could be affected by c.2677G>T, A, an SNP at exon 21 producing Ala893Thr and Ala893Ser, respectively, which is partially linked to c.3435C>T. Tanabe et al. (see for example, Tanabe, M., Ieiri, I., Nagata, N., Inoue, K., Ito, S., Kanamori, Y., Takahashi, M., Kurata, Y., Kigawa, J., Higuchi, S., Terakawa, N., and Otsubo, K. Expression of P-glycoprotein in human placenta: relation to genetic polymorphism of the multidrug resistance (MDR)-1 gene. J Pharmacol Exp Ther, 297: 1137-1143, 2001) reported that P-gp expression levels in placenta were affected by c.2677G>T, A. Thus the relationship between the c.3435C>T genotype and biochemical phenotypic P-gp activity appears not to be clearly established as pointed recently (see for example, Sakaeda, T., Nakamura, T., and Okumura, K. Pharmacogenetics of MDR1 and its impact on the pharmacokinetics and pharmacodynamics of drugs. Pharmacogenomics, 4: 397-410, 2003).

Furthermore, the following methods have been proposed: a method for estimating the side effect of immunosuppressive agent such as tacrolimus or cyclosporine, by investigating whether the 2677th base is guanine, or adenine or thymine, in the position of the cDNA sequence-coding region of human MDR1 gene (see for example, Japanese Laid-Open Patent Application No: 2002-223769); a method for diagnosing etiopathogenic by estimating the expression state of downstream genes such as IL-1α gene, PAI-1 gene, MDR1 gene, MMP-3 gene that are affected by the functional change of p53 gene, by investigating if the functional change caused by p53 gene is related to the development of cancer, in a cancer developed by the impairment of p53 gene function (see for example, Japanese Laid-Open Patent Application No: 2002-269).

ABC transporter is a target molecule playing an important role for susceptibility of anticancer agent or internal kinetics, and it is important to reveal the molecular background caused by individual difference of its expression, to perfom personalized treatment. Because many drugs are substrates of P-gp, degree of expression and activity of P-gp can directly affect the therapeutic effectiveness of such agents. Besides pharmacological relevance, inter-individual variety of P-gp SNPs and expression level may have another clinical impact as follows. Recently, the present inventors found the role of P-gp in colorectal carcinogenesis in mice (see for example, Mochida, Y, Taguchi, K., Taniguchi, S., Tsuneyoshi, M., Kuwano, H., Tsuzuki, T., Kuwano, M., and Wada, M. The role of P-glycoprotein in intestinal tumorgenesis: disruption of mdr1a suppresses polyp formation in Apc Min/+mice. Carcinogenesis, 24: 1219-1224, 2003; Yamada, T., Mori, Y., Hayashi, R., Takada, M., Ino, Y., Naishiro, Y., Kondo, T., and Hirohashi, S. Suppression of intestinal polyposis in Mdr1-deficient ApcMin/+ mice. Cancer Res, 63: 895-901, 2003). The present inventors found that DNA damage was significantly increased in mice disrupted in mdr1a, ortholog of human MDR1, compared with wild-type mice. Surprisingly, the present inventors also found that statistically smaller numbers of polyps were generated in mdr1a-disrupted mice compared with wild-type mice under APCMin background. Inter-individual variety of P-gp expression in colon could then be associated with colorectal carcinogenesis in human. The SNPs at the 5′ regulatory region of the human MDR1 gene are associated with the expression of MDR1 mRNA and P-gp in colorectal mucosa and liver in the Japanese population. The results would provide a framework for further analysis of the relationship between the SNPs of MDR1 and drug response, and as well as for further assessment of the importance of P-gp in inter-individual variability of drug response and cancer risk. Further, as it is estimated that MDR1 gene is associated with the biologic defense by exclusion of foreign substances, it may be possible to estimate and diagnose the onset of inflammatory intestinal disease, pathology/disease caused by impairment of the biologic defense function by exclusion of foreign substances, pathology/disease depending on cell survival, anti apoptosis function, or the development of pathology/disease due to impairment thereof. Further, by drug development targeting MDR1 beyond the estimation/diagnosis, it is possible to apply the drug to the prevention and treatment of the above disease.

Further knowledge of genotypic variation of the 5′ regulatory region of the MDR1 gene such as that provided by the present invention is useful and provides an advancement in the art which may facilitate, for example, estimating drug response and variation thereof, assessing drug pharmacokinetics, determining pharmacokinetic variability of drugs among individuals, providing personalized and individualized therapies, assessing cancer-related drug resistance, assessing oncogenic risk, and understanding underlying inter-individual variations in MDR1 expression, and additional similar benefits.

Citation or identification of any document in this application is not an admission that such document is available as prior art to the present invention.

### SUMMARY OF THE INVENTION

The present invention relates to methods for determining the genotype of a MDR1 gene that may comprise the step of detecting and/or determining the presence and/or identity of single nucleotide polymorphisms (SNPs) in an MDR1 gene. According to the present invention, SNPs occurring in the 5′ regulatory region of an MDR1 gene may be diagnostic for drug response and/or oncogenic risk. The SNPs may especially be in the 5′ regulatory region of a MDR1 gene. The instant invention further relates to determining haplotypes and/or diplotypes of the 5′ regulatory region of a MDR1 gene by steps that may include detecting SNPs in one or more nucleotide positions of the 5′ regulatory region. The SNPs of the present invention may be at one or more nucleotide positions in the 5′ regulatory region, for example, at one position or at two different positions. In another aspect, the present invention relates to previously unknown SNPs identified in the 5′ regulatory region of the MDR1 gene that may be useful as markers for diagnostics for purposes such as, for example, estimating and/or predicting drug response and/or assessing or estimating the onset of cancer, for example, colon cancer. The present invention also relates to primers and primer sets that may be used in the methods of the invention, for example, to hybridize to the MDR1 gene and detect one or more SNPs in the 5′ regulatory region of MDR1. In yet another aspect, the instant invention relates to a DNA of the 5′ regulatory region of a MDR1 gene that may comprise one or more SNPs. The instant invention also relates to a method for estimating the onset of colon cancer by steps that may comprise determining haplotypes and/or diplotypes of the 5′ regulatory region of an MDR1 gene or determining diplotype of the 5′ regulatory region of MDR1 gene.

Thus, an object of the present invention is to provide a method for determining haplotypes or diplotypes of MDR1 gene especially with respect to the 5′ regulatory region of the MDR1 gene.

The present invention further relates to a method for determining haplotypes and/or diplotypes of a 5′ regulatory region of MDR1 gene, by steps that may include detecting a polymorphism at a position selected from −2903, −2410, −2352, −1910, −1717 and −1325, when the position is indicated in relation to a first base of translation start codon (ATG) which is set to +1, in a nucleotide sequence of a 5′regulatory region of a MDR1 gene (“1”).

The instant invention further relates to a method for determining haplotypes and/or diplotypes of a 5′regulatory region of MDR1 gene according to steps that may comprise detecting a single nucleotide polymorphism at a position selected from −934 and/or −692 position, in addition to SNPs at a position selected from −2903, −2410, −2352, −1910, −1717 and −1325, when the position is indicated in relation to a first base of translation start codon (ATG) which is set to +1, in a nucleotide sequence of a 5′ regulatory region of a MDR1 gene (“2”).

Another aspect of the present invention provide for a method for determining haplotypes and/or diplotypes of a 5′regulatory region of a MDR1 gene according to “1” or “2”, that may comprise the step of investigating whether the base at −2903 is thymine or cytosine (“3”).

In yet another aspect, the present invention relates to a method for determining haplotypes and/or diplotypes of a 5′regulatory region of MDR1 gene according to any one of “1” to “3”, that may comprise the step of investigating whether the base at −2410 is thymine or cytosine (“4”).

The instant invention further relates to a method for determining haplotypes and/or diplotypes of a 5′regulatory region of MDR1 gene according to any one of “1” to “4”, that may comprise the step of investigating whether the base at −2352 is guanine or adenine (“5”). Another aspect of the present invention encompasses a method for determining haplotypes and/or diplotypes of a 5′regulatory region of MDR1 gene according to any one of “1” to “5” that may comprise the step of investigating whether the base at −1910 is thymine or cytosine (“6”).

The present invention also relates to a method for determining haplotypes and/or diplotypes of a 5′regulatory region of MDR1 gene according to any one of “1” to “6”, that may comprise the step of investigating whether the base at −1717 is thymine or cytosine (“7”). Another aspect of the present invention relates to a method for determining haplotypes and/or diplotypes of a 5′regulatory region of MDR1 gene according to any one of “1” to “7” that may comprise the step of investigating whether the base at −1325 is guanine or adenine (“8”).

Moreover, the present invention relates to a DNA comprising the 5′ regulatory region of MDR1 gene, wherein bases at −2410, −2352, −1910, −934, −692 may be replaced with thymine, adenine, thymine, adenine, thymine, respectively, when the position is indicated in relation to a first base of translation start codon (ATG) which is set to +1, in a nucleotide sequence of a 5′regulatory region of MDR1 gene (“9”). Another aspect of the present invention relates to a DNA comprising the 5′regulatory region of MDR1 gene, wherein bases at −2410, −2352, −1910, −934, −692 may be replaced with cytosine, guanine, cytosine, guanine, cytosine, respectively, when the position is indicated in relation to a first base of translation start codon (ATG) which is set to +1, in a nucleotide sequence of a 5′regulatory region of MDR1 gene (“10”).

The present invention further relates to a DNA comprising the 5′ regulatory region of MDR1 gene, wherein bases at −2410, −2352, −1910, −934, −692 are replaced with cytosine, adenine, cytosine, guanine, cytosine, respectively, when the position is indicated in relation to a first base of translation start codon (ATG) which is set to +1, in a nucleotide sequence of a 5′regulatory region of MDR1 gene (“11”). The instant invention also relates to a DNA comprising the 5′ regulatory region of MDR1 gene, wherein bases at −2410, −2352, −1910, −934, −692 are replaced with thymine, adenine, thymine, guanine, thymine, respectively, when the position is indicated in relation to a first base of translation start codon (ATG) which is set to +1 in a nucleotide sequence of a 5′regulatory region of MDR1 gene (“12”).

Moreover, the present invention relates to a primer set or primers that may comprise a forward primer that may hybridize with a region upstream of a position for detecting polymorphism, and a reverse primer that may hybridize with a region downstream of a position for detecting polymorphism, which may be used for a method for determining haplotypes of a 5′ regulatory region of MDR1 gene for detecting a polymorphism at a position selected from −2903, −2410, −2352, −1910, −1717 and −1325, when the position is indicated in relation to a first base of translation start codon (ATG) which is set to +1, in a nucleotide sequence of a 5′regulatory region of MDR1 gene (“13”).

The present invention further relates to a method for determining the diplotype of the 5′ regulatory region of a MDR1 gene that may comprise the step of detecting a polymorphism at −2352, and at a position selected from −2410, −1910 and −692, when the position is indicated in relation to a first base of translation start codon (ATG) which is set to +1, in a nucleotide sequence of a 5′regulatory region of MDR1 gene (“14”).

Furthermore, the present invention relates to the method for determining the diplotype of the 5′ regulatory region of a MDR 1 gene according to “14”, wherein gene-typing is performed by a PCR-based assay, such as, for example, TaqMan® (APPLIED BIOSYSTEMS) (“15”). The present invention further relates to a probe and a primer set that may be used in a method for determining diplotype of the 5′ regulatory region of a MDR1 gene that may comprise the step of detecting a polymorphism at −2352, and at a position selected from −2410, −1910 and −692, when the position is indicated in relation to a first base of translation start codon (ATG) which is set to +1, in a nucleotide sequence of a 5′regulatory region of MDR1 gene (“16”).

The instant invention further relates to a probe and primer set according to “16”, that may be used in a method for determining diplotype of the 5′ regulatory region of a MDR1 gene by a PCR-based assay, such as, for example, TaqMan® (APPLIED BIOSYSTEMS) (“17”).

Another aspect of the present invention relates to a method for estimating an onset of colon cancer, wherein the method for determining haplotypes and/or diplotypes of 5′ regulatory region of MDR1 gene according to any one of “1” to “8”, or the method for determining diplotype of 5′regulatory region of MDR1 gene according to “14” or “15” may be used (“18”). The present invention further relates to a method for developing a drug for controlling MDR1 expression, wherein at least one position selected from −2903, −2410, −2352, −1910, −1717 and −1325, −934, −692 may be targeted, when the position is indicated in relation to a first base of translation start codon (ATG) which is set to +1, in a nucleotide sequence of a 5′regulatory region of MDR1 gene (“19”).

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF DRAWING

The following detailed description, given by way of example, but not intended to limit the invention solely to the specific embodiments described, may best be understood in conjunction with the accompanying drawings, in which:

FIG. 1 is a figure showing the positions of SNPs of the 5′regulatory region of MDR1 gene.

FIG. 2 is a figure showing the association between diplotypes at the 5′regulatory region and mRNA level of the MDR1 gene. A: Five polymorphisms (−2410, −2352, −1910, −934 and −692) were analyzed at the 5′ regulatory region and MDR1 mRNA levels were measured in 72 normal colorectal mucosa by real-time PCR. The 72 samples were divided according to their diplotypes: diplotype A (haplotypes 1/1), diplotype B (haplotypes 1/2), diplotype C (haplotypes 1/3) and diplotype D (haplotypes 2/2). The MDR1 mRNA level was normalized with the GAPDH mRNA level. B: Five polymorphisms (−2410, −2352, −1910, −934, −692) at the 5′ regulatory region were analyzed and MDR1 mRNA levels were measured in 43 normal liver tissues.

FIG. 3 is a figure showing the results of immunohistochemical staining of P-gp by antibody JSB-1.

Positive staining for P-gp is observed in the apical membrane tissue of the surface epithelium region. Solid arrowheads indicate P-gp staining by anti-P-gp antibody (JSB-1). The values of MDR1 mRNA level for each sample are shown.

FIG. 4 is a figure showing the detection results of protein binding to the 5′ regulatory region of MDR1 by electrophoretic mobility shift assay. The experiments were performed three times each and similar results were obtained. In FIG. 4, NE represents nuclear extract.

The nuclear extracts (1-2 μl of protein) incubated with ³²P-labeled oligonucleotide in binding buffer B (for −2352G>A; panel A) or binding buffer A (for −692T>C; panel B) were resolved by gel electrophoresis. A 10-fold excess of the unlabeled oligonucleotide (−2352G or −2352A) was added for the competition. The solid arrowhead indicates a retarded DNA-protein complex and the asterisk indicates the non-specific binding of nuclear proteins.

### DETAILED DESCRIPTION

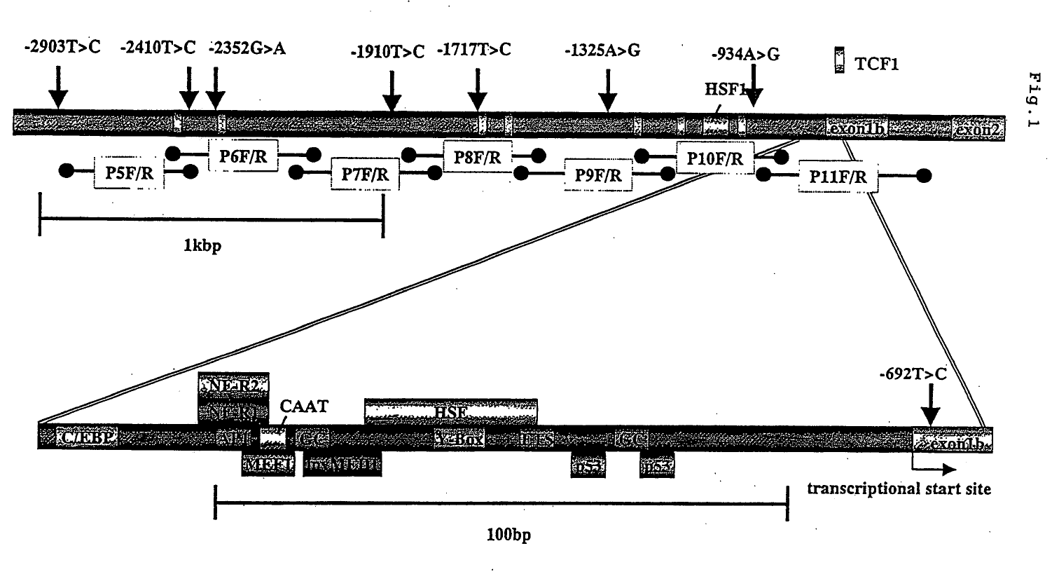

The present inventors analyzed the nucleotide sequence polymorphisms in the 5′ regulatory region of the gene spanning 4 kb from the transcriptional start site of MDR1 gene in the Japanese population, and identified eight single nucleotide polymorphisms (SNPs)(see FIG. 1). Of the eight SNPs identified, two (−692T>C and −934A>G) were known, while the six other SNPS (−1325A>G, −1717T>C, −1910T>C, −2352G>A, −2410T>C, and −2903T>C) were not reported and three among these (−1910T>C, −2352G>A, −2410T>C) were in perfect linkage disequilibrium. The present inventors found that haplotypes or diplotypes may be associated with the expression level of MDR1 gene in healthy colon mucous membrane; diplotypes wherein the expression level of MDR1 gene is estimated to be low were not observed in colon cancer patients; and on the contrary, the expression frequency of diplotypes wherein the expression level of MDR1 gene is estimated to be high, is higher in the colon cancer patient group than in the control group of healthy subjects; (from statistical analysis, by calculating odds ratio with the use of dyplotype A with high MDR1, diplotypes B and C with intermediate rate, diplotypes D and E with low MDR1, when diplotype A is set as 1, diplotypes D and E showed 0.524, a half level, and diplotypes B and C showed 0.892, an intermediate rate); the binding ability of a protein binding to the 5′ regulatory region of MDR1 gene may change significantly by SNPs; and that 95% or more may be converged into 3 haplotypes. Thus, in one aspect of the present invention, the present inventors obtained knowledge that SNPs of the 5′ regulatory region of the MDR1 gene may be useful as a marker for estimating drug response and oncogenic risk.

As for the method for determining haplotypes and/or diplotypes of the 5′regulatory region of MDR1 gene of the present invention, there is no particular limitation as long as it is a method for detecting a SNP. In one embodiment, the SNPs preferably occur at one or more positions selected from −2903, −2410, −2352, −1910, −1717 and −1325, where the position is indicated in relation to a first base of translation start codon ATG which is set to +1, in a nucleotide sequence of a 5′regulatory region of MDR1 gene. In another embodiment, the invention relates to a method for detecting one or more polymorphisms at −934 and/or −692, in addition to the above position selected from −2903, −2410, −2352, −1910, −1717 and −1325. In a further embodiment, the instant invention relates to a method for detecting one or more polymorphism at −2410, −2352, −1910, −934 and −692. Herein, the position to detect a polymorphism is indicated by a position in relation to the ATG start codon which is set to +1. ATG start codon is located at exon 2, and the transcription start site corresponds to −699 in this numbering system.

In an embodiment, the data obtained from gene typing experiments may be directly diplotypes, while it may be possible to estimate haplotypes reversely from the diplotypes. Embodiments relating to determining the expression level of the MDR1 gene or the risk of carcinogenesis can relate to or be based on diplotype data. Further, data obtained from gene typing by TaqMan method can be diplotypes.

The invention further relates to the complementary sequence of the 5′regulatory region of MDR1 gene (a part of GenBank accession nos. gi/19697556/gb/AC002457.2/, which is a complementary sequence information of the MDR1 gene region) as shown in SEQ ID NO:1. Therefore, it can be seen, for example, that complementary base “T” forming a base pair with the 2410th base “A” of SEQ ID NO:1 is the above −2410 base.

As for a method for detecting a polymorphism at a position selected from −2903, −2410, −2352, −1910, −1717, −1325, −934 and −692 mentioned above (hereinafter sometimes referred to as “predetermined substitution position”), specific examples may include: a method for detecting whether the base at −2903 is thymine or cytosine; a method for detecting whether the base at −2410 is thymine or cytosine; a method for detecting whether the base at −2352 is guanine or adenine; a method for detecting whether the base at −1910 is thymine or cytosine; whether the base at −1717 is thymine or cytosine; a method for detecting whether the base at −1325 is adenine or guanine; a method for detecting whether the base at −934 is adenine or guanine; a method for detecting whether the base at −692 is thymine or cytosine, respectively. SEQ ID NO:1 shows a normal complementary nucleotide sequence of a nucleotide sequence of 5′ regulatory region of MDR1 gene wherein bases at −2903, −2410, −2352, −1910, −1717, −1325, −934 and −692 are thymine, thymine, guanine, thymine, thymine, adenine, adenine, and thymine, respectively.

As for a method for detecting a polymorphism at a predetermined substitution position, there is no particular limitation as long as it is a method for detecting SNPs with the use of any appropriate conventionally known method, such as, but not limited to, PCR, ligand strings reactions, restriction enzyme digestion methods, a direct base sequencing analysis, nucleic acid amplification techniques, hybridization methods, immunoassays, mass spectrometry, etc. For example, the above method may be performed by the following methods: as the nucleotide sequence of the 5′regulatory region of MDR1 gene is already known (see SEQ ID NO:1), a method for directly sequencing with the use of a primer set (SEQ ID NOs: 2 to 25) comprising a forward primer that hybridizes with a region upstream of a predetermined substitution position (position for detecting polymorphism) shown in Table 1 in the following and a reverse primer that hybridizes with a region downstream of a predetermined position, and amplifying by known nucleic acid amplification methods such as PCR to determine the nucleotide sequence of the amplified fragment. A further method may be to use TaqMan. Still other methods may involve restriction fragment length polymorphism (RFLP) of the amplified fragment, SSCP (single-strand conformation polymorphism), ASO hybridization, ARMS method, denaturing gradient gel electrophoresis, RnaseA digestion method, chemical digestion method, DOL method, invader method, MALDI-TOF/MS method, TDI method, molecular beacon method, dynamic allele specific hybridization method, Padlock probe method, UCAN method, nucleic acid hybridization method by using DNA tip or DNA microarray, or ECA method. As long as the above primers have a size to hybridize specifically to the nucleotide sequence of the 5′regulatory sequence of MDR1 gene, the size of the primers is not particularly limited, and those having a size of 15 to 40 bases, preferably about 20 bases may be used, and there is no particular limitation for the size of an amplification regions. Further, genomic DNA as the PCR may be prepared by methods known in the art from a sample comprising viable cells, including, for example, living or dead cells or both, such as, for example, peripheral blood, hair root, oral mucosa, blood smear preparation, regardless of MDR1 expression.

Determination of haplotypes or diplotypes of the 5′regulatory region of MDR1 gene may be determined by detecting a polymorphism at all of the predetermined substitution positions, but it can be determined by detecting polymorphism at certain predetermined substitution positions. For example, when the frequency of minor allele detects a certain level of polymorphism at −2410, −2352, −1910, −934 and −692, haplotypes or diplotypes can be aggregated.

As for a DNA of the 5′ regulatory region of MDR1 gene of the present invention, reference to the nucleotide position is with respect to the first base of translation start codon ATG which is set to +1. A nucleotide sequence of a 5′regulatory region of MDR1 gene may comprise the base at −2410 replaced with thymine, the base at −2352 replaced with adenine, the base at −1910 replaced with thymine, the base at −934 position replaced with adenine, the base at −692 replaced with thymine. In another embodiment, the invention relates to a DNA wherein the base at −2410 is replaced with cytosine, the base at −2352 with guanine, the base at −1910 with cytosine, the base at −934 with guanine, the base at −692 with cytosine; or a DNA wherein the base at −2410 is replaced with cytosine, the base at −2352 with adenine, the base at −1910 with cytosine, the base at −934 with guanine, the base at −692 with cytosine. In yet another embodiment, a DNA is provided wherein the base at −2410 is replaced with thymine, the base at −2352 with adenine, the base at −1910 with thymine, the base at −934 with guanine, the base at −692 with thymine, can be exemplified. These DNAs may constitute a haplotype of the 5′regulatory region of MDR1 gene, for example, where the DNA comprises a thymine at position −2410, a guanine at −2352, a thymine at −1910, an adenine at −934, and a thymine at base at −692.

As for a primer set of the present invention, there is no particular limitation as long as it is a primer set used for a method for determining haplotypes of the 5′regulatory region of MDR1 gene by detecting a polymorphism at a position selected from −2903, −2410, −2352, 1910, −1717 and −1325, when the position is indicated in relation to a first base of translation start codon ATG which is set to +1, in a nucleotide sequence of a 5′regulatory region of MDR1 gene. In an embodiment, the primer set may comprise a forward primer that hybridizes with a region upstream of a position detecting polymorphism, and a reverse primer that hybridizes with a region downstream of a position for detecting polymorphism, and that each of these primers may have a size to hybridize specifically to the nucleotide sequence of the 5′ regulatory region of MRD1 gene. Generally, those primers having a size from 15 to 40 bases, preferably those of about 20 bases, may be used in the present invention. The size of an amplifying region may not be particularly limited, and specifically may include, for example, primer sets such as P5F/R that detects a polymorphism at −2410 (SEQ ID Nos: 10 and 11), P6F/R that detects a polymorphism at −2352 (SEQ ID Nos: 12 and 13), P7F/R that detects a polymorphism at −1910 (SEQ ID Nos: 14 and 15), P8F/R that detects polymorphism at −1717 (SEQ ID Nos: 16 and 17), and P9F/R that detects a polymorphism at −1325 (SEQ ID Nos: 18 and 19), as shown in Table 1.

Further, as for a method for determining diplotype at the 5′ regulatory region of MDR1 gene of the present invention, there is no particular limitation as long as it is a method for detecting polymorphism at −2352, and at a position selected from −2410, −1910 and −692, when the position is indicated in relation to a first base of translation start codon ATG which is set to +1, in a nucleotide sequence of a 5′regulatory region of MDR1 gene. As it is shown in Table 3 of Example 12 and described in the following, −2410T(C), −1910T(C) and −692T(C) are usually detected together in each clone, due to linkage disequilibrium. However, as −2352G(A) is independent, a method for detecting a polymorphism at −2352 and −2410, a method for detecting a polymorphism at −2352 and −1910, or a method for detecting a polymorphism at −2352 and −692 may be specifically exemplified. For example, it is possible to detect a polymorphism at −2352G(A) and −692T(C), and to determine/classify into diplotypes such that (−2352, −692, −2352, −692) is (G,T/G,T), (G,T/A,T), (G,T/G,C), (A,T/A,T), or (G,C/G,C).

As for the method for determining diplotype of the 5′ regulatory region of MDR1 gene, a method performing gene typing by TaqMan method may be advantageously exemplified. As for a probe/primer set used for performing gene typing by TaqMan method, for −2352 typing, a probe for detecting G shown in SEQ ID NO:61, a probe for detecting A shown in SEQ ID NO:62, and a primer set shown in SEQ ID Nos: 63 and 64; and for −692 typing, a probe for detecting T shown in SEQ ID NO:69, a probe for detecting C shown in SEQ ID NO:70, a primer set shown in SEQ ID Nos: 71 and 72 may be advantageously exemplified.

As it is describe in the above, since diplotypes, which are expected to have a high expression level of MDR1 gene, appear more frequently in the colon cancer patient group than in the normal healthy control group, (from statistical analysis, by calculating odds ratio with the use of dyplotype A with high MDR1, diplotypes B and C with intermediate rate, diplotypes D and E with low MDR1, when diplotype A is set as 1, diplotypes D and E showed 0.524, and diplotypes B and C showed 0.892, an intermediate rate), it may be possible to estimate the onset of colon cancer by using a method for determining haplotypes and/or diplotypes of the 5′regulatory region of MDR1 gene of the present invention, or by a method for determining diplotype of the 5′regulatory region of MDR1 gene. Further, it may be possible to develop agents for controlling MDR1 expression targeting at least one position selected from −2903, −2410, −2352, −1910, −1717 and −1325, −934, −692, when the position is indicated in relation to a first base of translation start codon ATG which is set to +1, in a nucleotide sequence of a 5′regulatory region of MDR1 gene.

In the following, the present invention will be explained in detail with reference to the examples, while the technical scope of the present invention will not be limited to these examples.

### EXAMPLE 1

Blood samples were obtained from 25 healthy Japanese volunteers at Kyushu University. Clinical samples of normal colorectal mucosa were taken from 72 Japanese patients who had undergone surgical resection of the cancer at the Department of Surgery II, Kyushu University Hospital (Fukuoka, Japan), the Coloproctology Center, Takano Hospital (Kumamoto, Japan) or the Department of Surgery I, Gunma University Hospital (Maebashi, Japan) between September 1993 and August 1998. These samples of blood and noncancerous mucosa were obtained under an Institutional Review Board (IRB)-approved protocol, with all subjects providing their informed consent. The samples were frozen in liquid nitrogen and stored at −80° C. until RNA and DNA were extracted. None of patients had received chemotherapy before the surgical resection. Clinical samples of normal liver tissues were taken as a non-cancerous tissue surrounding cancer from 43 Japanese patients with hepatocellular carcinoma, who had undergone surgical resection of cancer at the Department of Surgery II, Kyushu University Hospital (Fukuoka, Japan).

### EXAMPLE 2

Genomic DNA from the volunteers' blood samples was isolated using the QiaAmp (Qiagen) blood kits, and DNA from tissues of patients was isolated using the Easy DNA Kit (Invitrogen) according to the manufacturer's protocol. RNA was isolated using the RNA extraction reagent TRIzol (Invitrogen Life Technologies) or Rneasy (Qiagen) according to the respective manufacturers' protocols.

### EXAMPLE 3

Specific oligonucleotide primers for PCR amplification of MDR1 gene fragments were derived from known sequences [GenBank accession nos.: AC002457 for the 5′ regulatory region and exons 1-7 and AC005068 for exons 8-28]. The locations of the SNPs in the exons corresponded to positions of the MDR1 cDNA (GenBank accession no. M14758, codon TTC in exon 10, F335, is missing in that sequence), which the first base of the ATG start codon was set to +1. The exons were defined by Chen et al. (Chen, C. J., Clark, D., Ueda, K., Pastan, I., Gottesman, M. M., and Roninson, I. B. Genomic organization of the human multidrug resistance (MDR1) gene and origin of P-glycoproteins. J Biol Chem, 265: 506-514, 1990), and the Human Genome Organisation recommended nomenclature (Antonarakis, S. E. Recommendations for a nomenclature system for human gene mutations. Nomenclature Working Group. Hum Mutat, 11: 1-3, 1998) was used for SNP nomenclature. The primers were designed to amplify the regions that include sequences including the SNPs reported previously, or to cover about 4 kb of the MDR1 upstream regulatory region as shown in Table 1. The PCR conditions for these primers are available by a request to the present inventors. Sequences of purified PCR fragments were obtained by automated DNA sequencing on ABI3700 (capillary) sequencers by using BigDye Terminator cycle sequencing reactions (Perkin-Elmer).

### EXAMPLE 4

Haplotypes of individuals who were heterozygous at least in one SNP locus were determined by PCR amplification and sequencing, using the forward primer MDR1P5F and the reverse primer MDR1P11R. Nucleotide sequences (SEQ ID Nos: 2 to 25) of the 12 primer sets used for screening SNPs of the 5′ regulatory region of MDR1 gene spanning for about 4-kb are shown in Table 1. PCR amplification was performed by using high fidelity DNA polymerase, KOD-Plus (Toyobo), according to the manufacture's protocol. The fragments were inserted into pT7Blue-3 vector (Novagen) and subcloned. At least six colonies were picked up, and plasmids were purified by the Qiagen DNA kit according to the manufacturer's protocol. SNP sites were analyzed by sequencing, and haplotypes were confirmed.

TABLE 1

| Sequences of oligonucleotide primers used for direct sequencing | |
|---|---|
| | Product |
| | length |
| Primer pair | (bp) |
| MDR1P1F: TATATGTCTCAGCCTGGGCG | 324 |
| MDR1P1R: TCACAGGAGAGCAGACACGT | |
| MDR1P2F: CTCTTGCTCACTCTAGGGAC | 227 |
| MDR1P2R: CAAATATGATCATGAGCCAC | |
| MDR1P3F: CACATATCATCTGAGAAGCCCA | 233 |
| MDR1P3R: AGGACACACCACTTCACTGC | |
| MDR1P4F: AGGCAGTGAAGTGGTGTGTC | 453 |
| MDR1P4R: ACCTTCATTCAAGCGGTGAT | |
| MDR1P5F: ATGAGAGCGGAGGACAAGAA | 469 |
| MDR1P5R: AACCCTCCCTAAACAGTGCA | |
| MDR1P6F: GAGATCTTTACCTGATGCTCA | 355 |
| MDR1P6R: AGGCTTCTAACAGGCCACTA | |
| MDRLP7F: AACAATGCTGTACACTTGCA | 443 |
| MDR1P7R: CTTGGCCTTACAATACAATG | |
| MDR1P8F: CGACAAAGCAAGACTCCGTC | 438 |
| MDR1P8R: CCTTCCATATTTACTGCCAACA | |
| MDR1P9F: GAATTGTGCAGATTGCACG | 437 |
| MDR1P9R: TCCGACCTCTCCAATTCTGT | |
| MDR1P10F: AGCATGCTGAAGAAAGACCA | 380 |
| MDR1P10R: TCAGCCTCACCACAGATGAC | |
| MDR1P11F: CTCGAGGAATCAGCATTCAG | 472 |
| MDR1P11R: GTCCAGTGCCACTACGGTTT | |
| MDR1P12F: GGGACCAAGTGGGGTTAGAT | 474 |
| MDR1P12R: CTTCTTTGCTCCTCCATTGC | |

The nucleotide sequences of 12 primer pairs used to screen the SNPs at the 5′ regulatory region of the MDR1 gene spanning about 4 kb.

### EXAMPLE 5

Quantitative RT-PCR was performed by real-time Taqman® technology and Model 7900 Sequence Detectors (Perkin-Elmer) as described previously (Gibson, U. E., Heid, C. A., and Williams, P. M. A novel method for real time quantitative RT-PCR. Genome Res, 6: 995-1001, 1996). The sequences of the primer pairs and the probe used in this study were described previously (Hinoshita, E., Uchiumi, T., Taguchi, K., Kinukawa, N., Tsuneyoshi, M., Maehara, Y., Sugimachi, K., and Kuwano, M. Increased expression of an ATP-binding cassette superfamily transporter, multidrug resistance protein 2, in human colorectal carcinomas. Clin Cancer Res, 6: 2401-2407, 2000).

### EXAMPLE 6

To extract type 3 allele, fragments including −2604 to −570 were amplified from templates corresponding to homozygotes for haplotypes 1 and 2, and to heterozygotes for haplotypes 1 and 3. The forward primer 5′-AAAGCTAGCTGTCAGTGGAGCAAAGAAATG-3′ (SEQ ID No: 26) and the reverse primer 5′-AAAGCTAGCCTCGCGCTCCTTGGAA-3′ (SEQ ID No: 27), each of which included an NheI site, were used. These amplification products were inserted into the NheI site of a pGL3 Basic vector (Promega). SNP sites in the constructs were confirmed by sequencing.

### EXAMPLE 7

Human hepatocarcinoma cell line (HepG2) was used in this study. Cells were grown at 37° C. in a humidified atmosphere containing 5% carbon dioxide. A total of 1 μg pGL3-Basic Vector DNA or reporter construct was transfected, and then, 100 ng phRL-TK Vector DNA (Promega) was co-transfected in all wells as a transfection control, by using LIPOFECTAMINE 2000 (Life Technologies) reagent and according to the manufacture's protocol. The plates were incubated at 37° C. for 6 hr after adding DNA—LIPOFECTAMINE complex, and the growth medium was then changed. The plates were incubated for further 24 hr prior to luciferase assay. Firefly and renilla luciferase activities were measured in a luminometer using the Dual-Luciferase Reporter Assay System (Promega). Data were normalized for transfection efficiency by the Renilla luciferase activity. In all cases, transfections were carried out in triplicate, with 3 wells of a 24-well plate containing identical transfection reactions.

### EXAMPLE 8

The primary antibodies used were P-gp (JSB-1) (mouse monoclonal, Sanbio). Immunostaining of P-gp was performed as described previously. To assure quantitative detection of P-gp by immunohistochemistry, an additional marker protein that is expressed in enterocytes, villin, was used. For quantification, ImageGauge (Fuji Photo Film Co.) software was used.

### EXAMPLE 9

The DNA sequences of the sense strand of each oligonucleotide were 5′-AAATGAAAGGTGAGATAAAGCAACAA-3′ (−2352G; SEQ ID No: 28), 5′-AAATGAAAGGTGAAATAAAGCAACAA-3′ (−2352A; SEQ ID No: 29), 5′-GAGCTCATTCGAGTAGCGGCTCTTCC-3′ (−692T; SEQ ID No: 30), and 5′-GAGCTCATTCGAGCAGCGGCTCTTCC-3′ (−692C; SEQ ID No: 31). Nuclear extracts (2 μg/μl of protein) were prepared from HepG2 cells as described previously. They were then incubated for 30 min at room temperature in a final volume of 10 μl of reaction mixture containing 2 μl of 5× binding buffer; 5 mM DTT; 10 ng of poly (dI-dC); and 1×10⁴ cpm of ³²P-labeled oligonucleotide probe in the absence or presence of various competitors. The present inventors tried five different kind of binding buffer, determined the one that generated clearest retarded band and used for further analyses. The composition of the 5× binding buffers used for the detailed analyses were as follows: Buffer A, 60 mM HEPES, 300 mM KCl, 20 mM MgCl₂, 5 mM EDTA, 60% (v/v) glycerol; Buffer B, 50 mM Tris-HCl (pH 7.5), 250 mM NaCl, 12.5 mM CaCl₂, 5 mM EDTA, 40% (v/v) glycerol. Next, the samples were electrophoresed on 4% polyacrylamide gel (polyacrylamide/bisacrylamide ratio, 79:1) in a Tris-borate-EDTA buffer (0.089 M Tris, 0.089 M Boric acid and 0.002 M EDTA). The gel was exposed to an imaging plate and analyzed using a Fujix BAS 2000 bioimage analyzer (Fuji Photo Film Co.).

### EXAMPLE 10

Statview 5.0 software (SAS Institute) was used for statistical analysis. Results of MDR1 mRNA levels versus diplotypes were analyzed by the Kruskal-Wallis test. Significance was defined as p<0.05. The correlations between MDR1 mRNA level and P-gp level were determined using Spearman's test. This test is usually used for nonparametric analysis when it is unclear whether or not the variables show normal distribution. Probability values of less than 0.05 were considered significant. The Spearman's coefficient I and associated probability (P) were calculated. Unpaired t-tests were performed to compare relative luciferase activities of reporter constructs containing haplotype 1, 2, or 3 at the 5′ regulatory region of MDR1 gene in transfection experiments.

### EXAMPLE 11

To find MDR1 polymorphisms at the 5′ regulatory region, genomic DNA isolated from peripheral blood of 25 healthy Japanese volunteers was analyzed. The upstream region, spanning about 4 kb from the transcriptional start site, was amplified by PCR and analyzed by direct sequencing. Eight SNPs were identified at the 5′ regulatory region, and six of them had not been reported before. The allele frequencies of these SNPs observed in the 50 chromosomes are presented in Table 2. The ATG start codon locates in exon2 and the transcription start site corresponds to −699 from the ATG in the genomic DNA. SNPs −692T>C and −934A>G were identical to the previous reported −129T>C and −41aA>G (Tanabe, M., Ieiri, I., Nagata, N., Inoue, K., Ito, S., Kanamori, Y., Takahashi, M., Kurata, Y., Kigawa, J., Higuchi, S., Terakawa, N., and Otsubo, K. Expression of P-glycoprotein in human placenta: relation to genetic polymorphism of the multidrug resistance (MDR)-1 gene. J Pharmacol Exp Ther, 297: 1137-1143, 2001), respectively, by this numbering system.

The sequences were inspected for deviations from the original MDR1 sequences (GenBank accession nos.: AC002457; AC005068), which we defined as the major-type. The eight SNPs (SEQ ID Nos: 32 to 47) of the 5′ regulatory region and the other six SNPs in exons and introns were analyzed: these six SNPs were previously reported to have allele frequencies of more than 0.05 in Caucasians as well as in Japanese (Hoffineyer, S., Burk, O., von Richter, O., Arnold, H. P., Brockmoller, J., Johne, A., Cascorbi, I., Gerloff, T., Roots, I., Eichelbaum, M., and Brinkmann, U. Functional polymorphisms of the human multidrug-resistance gene: multiple sequence variations and correlation of one allele with P-glycoprotein expression and activity in vivo. Proc Natl Acad Sci USA, 97: 3473-3478, 2000; Ito, S., Ieiri, I., Tanabe, M., Suzuki, A., Higuchi, S., and Otsubo, K. Polymorphism of the ABC transporter genes, MDR1, MRP1 and MRP2/cMOAT, in healthy Japanese subjects. Pharmacogenetics, 11: 175-184, 2001; and Tanabe, M., Ieiri, I., Nagata, N., Inoue, K., Ito, S., Kanamori, Y., Takahashi, M., Kurata, Y., Kigawa, J., Higuchi, S., Terakawa, N., and Otsubo, K. Expression of P-glycoprotein in human placenta: relation to genetic polymorphism of the multidrug resistance (MDR)-1 gene. J Pharmacol Exp Ther, 297: 1137-1143, 2001). The allele frequencies of these SNPs obtained from analysis of the present inventors are shown in Table 2. The frequencies in Table 2 were calculated from the results of genomic DNA analysis of peripheral blood of SNPs of the 5′ regulatory region, coding region and intron region, obtained from 25 healthy volunteers. The allele frequencies were within the range expected from sample size as those reported before. A strong association between c.3435C>T and c.2677G>T, A was observed as previously reported (Tanabe, M., Ieiri, I., Nagata, N., Inoue, K., Ito, S., Kanamori, Y, Takahashi, M., Kurata, Y., Kigawa, J., Higuchi, S., Terakawa, N., and Otsubo, K. Expression of P-glycoprotein in human placenta: relation to genetic polymorphism of the multidrug resistance (MDR)-1 gene. J Pharmacol Exp Ther, 297: 1137-1143, 2001; Kim, R. B., Leake, B. F., Choo, E. F., Dresser, G K., Kubba, S. V., Schwarz, U. I., Taylor, A., Xie, H. G., McKinsey, J., Zhou, S., Lan, L. B., Schuetz, J. D., Schuetz, E. G. and Wilkinson, G R. Identification of functionally variant MDR1 alleles among European Americans and African Americans. Clin Pharmacol Ther, 70: 189-199, 2001), whereas there was no linkage between the polymorphisms at the 5′ regulatory region and those of coding region including c.1236C>T, c.2677G>T, A and c.3435C>T. No association between −692T>C and c.2677G>T, A nor c.3435C>T is consistent with previous reports (Kim, R. B., Leake, B. F., Choo, E. F., Dresser, G. K., Kubba, S. V., Schwarz, U. I., Taylor, A., Xie, H. G., McKinsey, J., Zhou, S., Lan, L. B., Schuetz, J. D., Schuetz, E. G., and Wilkinson, G R. Identification of functionally variant MDR1 alleles among European Americans and African Americans. Clin Pharmacol Ther, 70: 189-199, 2001; Horinouchi, M., Sakaeda, T., Nakamura, T., Morita, Y., Tamura, T., Aoyama, N., Kasuga, M., and Okumura, K. Significant genetic linkage of MDR1 polymorphisms at positions 3435 and 2677: functional relevance to pharmacokinetics of digoxin. Pharm Res, 19: 1581-1585, 2002).

TABLE 2

Frequencies of SNPs in the MDR1 gene in the
Japanese population

| | | | |
|---|---|---|---|
| | Nucleic acid | | Allele |
| | substitution | Amino acid | frequency^{B} |
| Location^{A} | (major/minor) | substitution | (major/minor) |

5′ regulatory region

| | | |
|---|---|---|
| −2903T>C | AGAGTATAG/ | 0.98/0.02 |
| −2410T>C | AGAGCATAG | 0.90/0.10 |
| −2352G>A | AGGGTTTAA/ | 0.72/0.28 |
| −1910T>C | AGGGCTTAA | 0.90/0.10 |
| −1717T>C | GTGAGATAA/ | 0.98/0.02 |
| −1325A>G | GTGAAATAA | 0.98/0.02 |
| −934A>G | ATGGTGTGA/ | 0.90/0.10 |
| −692T>C | ATGGCGTGA | 0.90/0.10 |
| | ATTATGGCT/ | |
| | ATTACGGCT | |
| | CTGGAAAAA/ | |
| | CTGGGAAAA | |
| | CCCAATGAT/ | |
| | CCCAGTGAT | |
| | CGAGTAGCG/ | |
| | CGAGCAGCG | |

Coding region

| | | | |
|---|---|---|---|
| c.1236 | AGGGCCTGA/ | Gly412Gly | 0.66/0.34 |
| C>T | AGGGTCTGA | Ala893Ser | 0.50/0.36 |
| (exon 12) | | | |
| c.2677 | AGGTGCTGG/ | Ala893Thr | /0.14 |
| C>T, A | AGGTTCTGG | Ile1145Ile | 0.58/0.42 |
| (exon 21) | | | |
| c.3435 | / | | |
| C>T | AGGTACTGG | | |
| (exon 26) | | | |
| | AGATCGTGA/ | | |
| | AGATTGTGA | | |

Intronic region

| | | |
|---|---|---|
| IVS4-25 | AATGGTATG/ | 0.96/0.04 |
| G>T | AATGTTATG | 0.52/0.48 |
| (intron 4) | | |
| IVS6+139 | GCAACAATG/ | 0.64/0.36 |
| C>T | GCAATAATG | |
| (intron 6) | | |
| IVS16-76 | TTACTAATT/ | |
| T>A | TTACAAATT | |
| (intron 16) | | |

^{A}The locations of the SNPs correspond to positions of the MDR1 cDNA, with the first base of the ATG start codon set to +1. The ATG start codon locates in exon2 and the transcription start site corresponds to −699 in this numbering system.
^{B}Frequency was calculated from the results of genomic DNA analysis of the peripheral blood of 25 healthy volunteers for the SNPs at the 5′ regulatory region as well as at the coding and intronic regions.

### EXAMPLE 12

To unequivocally determine the frequency of haplotypes at the regulatory region, the 2 kb fragment containing these polymorphic sites at the −2410, −2352, −1910, −934, and −692 was amplified by PCR. Of 25 blood samples, analysis of homozygous samples at all these sites was omitted, and heterozygous samples were used at least in one of those sites. After subcloning the amplified fragments into the pT7Blue3 vector, their nucleotide sequences were determined. Since the frequencies of the minor alleles at −2903, −1717 and −1325 were too low (0.02) for statistical analysis, these three alleles from the analysis were omitted. The frequencies were calculated from the genetic type of 25 samples of healthy Japanese volunteers, wherein fragments corresponding to the region were confirmed by PCR amplification and sequencing. As shown in Table 3, −2410T(C), −1910T(C) and −692T(C) were detected together in each clone, but −2352G(A) was independent. The haplotypes were determined as follows: haplotype 1 (−2410T, −2352G, −1910T, −934A, −692T), haplotype 2 (−2410T, −2352A, −1910T, −934A, −692T) and haplotype 3 (−2410C, −2352G, −1910C, −934G, −692C). Three haplotypes (haplotypes 1, 2 and 3) accounted for more than 95% of the population. The promoter haplotypes were not associated with any SNPs examined in coding and intron regions in Japanese.

TABLE 3

Haplotypes at the 5′ upstream regulatory region
of MDR1

| | | | | | | |
|---|---|---|---|---|---|---|
| haplo- | | | | | | |
| type | −2410 | −2352 | −1910 | −934 | −692 | Frequency (%) |

| | | | | | | |
|---|---|---|---|---|---|---|
| 1 | T | G | T | A | T | 64 |
| 2 | T | A | T | A | T | 24 |
| 3 | C | G | C | G | C | 8 |
| 4 | C | A | C | G | C | 2 |
| 5 | T | A | T | G | T | 2 |

Frequency was calculated from the genotyping of 25 samples of healthy Japanese volunteers confirmed by PCR amplification and sequencing of corresponding fragments in the region.

### EXAMPLE 13

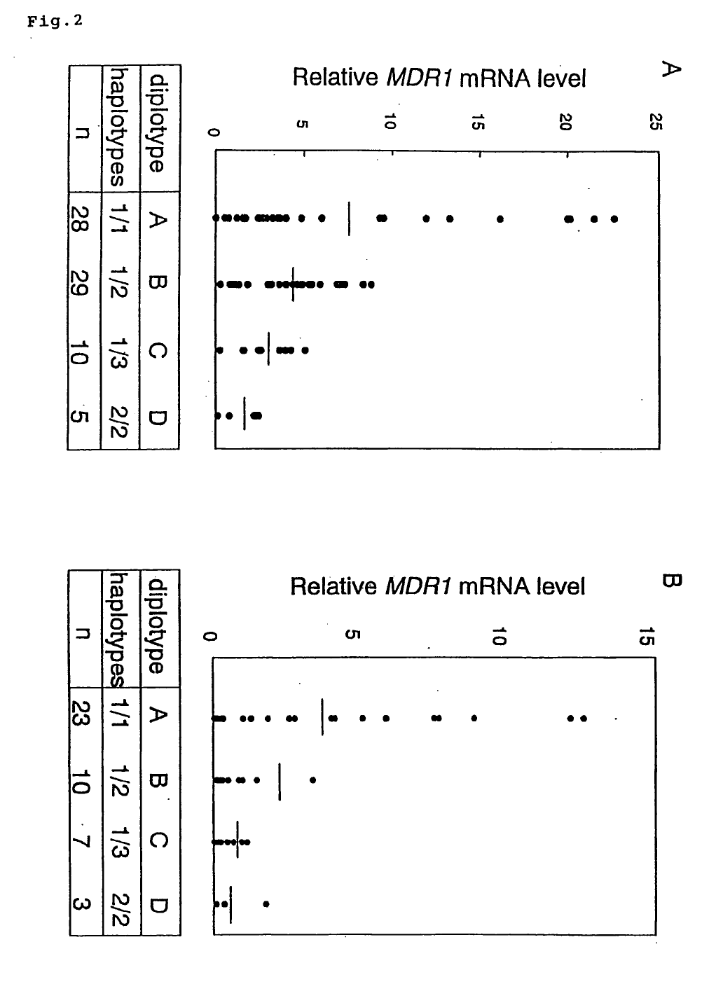

Each of the five polymorphisms (−2410, −2352, −1910, −934, −692) at the 5′ regulatory region for any association with MDR1 mRNA levels in 72 normal colorectal mucosa was tested. The 72 samples were divided according to diplotype into four groups: diplotype A (haplotypes 1/1), diplotype B (haplotypes 1/2), diplotype C (haplotypes 1/3) and diplotype D (2/2). Then, it found that the mean MDR1 mRNA level of diplotype A, which had two haplotype 1, was higher than that of diplotype D, which did not have haplotype 1 (p=0.04) (FIG. 2A). The mean MDR1 mRNA levels of diplotypes B and C, each of which had one haplotype 1, were intermediate between those of diplotypes A and D. MDR1 mRNA level was normalized with GAPDH mRNA level. When MDR1 mRNA levels in the samples from normal liver were analyzed, results were similar to those from colon (FIG. 2B), although the association was statistically much lower compared to that in colon (p=0.2).

Further, each SNP was analyzed for any individual associations with mRNA level. The association between −692T>C and mRNA level in colon was not statistically significant in the present study, although there was a tendency toward lower mRNA levels in T/C heterozygotes than in T/T homozygotes (p=0.2, data not shown). For −2352G>A, A/A homozygotes showed lower mRNA levels than in G/G homozygotes (p=0.07).

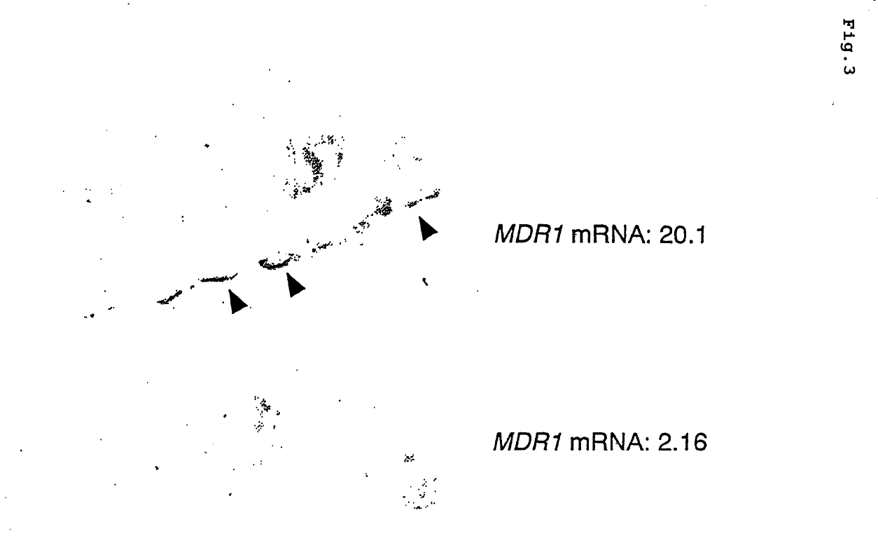

Then, a correlation of mRNA expression levels of MDR1 and the expression levels of P-gp was confirmed. Since a sufficient volume of each sample for Western blotting was not available, P-gp levels were measured by immunohistochemistry method using antibody JSB-1. Therefore, the measurements were semi-quantitative rather than quantitative. 14 of the 72 samples were examined and it was found that MDR1 mRNA level showed a significant correlation with P-gp level by Spearman's test (r=0.428, p=0.01). Representative data is presented in FIG. 3.

c.3435C>T, which was reported to affect P-gp level in intestine and/or function (Hoffineyer, S., Burk, O., von Richter, O., Arnold, H. P., Brockmoller, J., Johne, A., Cascorbi, I., Gerloff, T., Roots, I., Eichelbaum, M., and Brinkmann, U. Functional polymorphisms of the human multidrug-resistance gene: multiple sequence variations and correlation of one allele with P-glycoprotein expression and activity in vivo. Proc Natl Acad Sci USA, 97: 3473-3478, 2000; Nakamura, T., Sakaeda, T., Horinouchi, M., Tamura, T., Aoyama, N., Shirakawa, T., Matsuo, M., Kasuga, M., and Okumura, K. Effect of the mutation (C3435T) at exon 26 of the MDR1 gene on expression level of MDR1 messenger ribonucleic acid in duodenal enterocytes of healthy Japanese subjects. Clin Pharmacol Ther, 71: 297-303, 2002), was not associated with the MDR1 mRNA level in colon and liver in this study (p=0.7), consistent with the previous report analyzing intestine (Goto, M., Masuda, S., Saito, H., Uemoto, S., Kiuchi, T., Tanaka, K., and Inui, K. C3435T polymorphism in the MDR1 gene affects the enterocyte expression level of CYP3A4 rather than Pgp in recipients of living-donor liver transplantation. Pharmacogenetics, 12: 451-457, 2002) or placenta (Tanabe, M., Ieiri, I., Nagata, N., Inoue, K., Ito, S., Kanamori, Y., Takahashi, M., Kurata, Y., Kigawa, J., Higuchi, S., Terakawa, N., and Otsubo, K. Expression of P-glycoprotein in human placenta: relation to genetic polymorphism of the multidrug resistance (MDR)-1 gene. J Pharmacol Exp Ther, 297: 1137-1143, 2001). c.2677G>T, A, which was also reported to correlate with the MDR1/P-gp expression level in placenta (Tanabe, M., Ieiri, I., Nagata, N., Inoue, K., Ito, S., Kanamori, Y., Takahashi, M., Kurata, Y., Kigawa, J., Higuchi, S., Terakawa, N., and Otsubo, K. Expression of P-glycoprotein in human placenta: relation to genetic polymorphism of the multidrug resistance (MDR)-1 gene. J Pharmacol Exp Ther, 297: 1137-1143, 2001), was not associated with the MDR1 mRNA level in colon and liver in the present invention (p=0.89), consistent with the results analyzing mRNA level in intestine (the above Pharmacogenetics, 12: 451-457, 2002). None of other SNPs examined in this invention was associated with MDR1 mRNA level in either colorectal epithelium or liver.

### EXAMPLE 14

To 443 colon cancer patients and 758 of general population of the area in the North region of Kyushu, after explaining them the research purposes and obtaining their informed consent, 10 cc of peripheral blood was collected, and typing was performed by TaqMan method with the use of ABI7900HT (Applied Biosystems) using the extracted genomic DNA as a template. The results are shown in Table 4. As it is clear from Table 4, diplotype E is observed in 9 subjects in control group (healthy subjects), while none was observed in colon cancer patients. Further, by calculating odds ratio with the use of dyplotype A with high MDR1 expression level, diplotypes B and C with intermediate rate, diplotypes D and E with low MDR1, when diplotype A was set to 1, diplotypes D and E showed 0.524 about half rate, and diplotypes B and C showed 0.892, an intermediate rate. From these results, it can be estimated that the risk of colon cancer onset is reduced by half for diplotypes D and E, compared to diplotype A.

TABLE 4

| | | | Colon cancer |
|---|---|---|---|
| Diplotype | −2352, −692 | Control (%) | patient cases (%) |

| | | | | | |
|---|---|---|---|---|---|
| A | G, T/G, T | 369 | (48.7) | 234 | (52.8) |
| B | G, T/A, T | 266 | (35.1) | 153 | (34.5) |
| C | G, T/G, C | 73 | (9.6) | 39 | (8.8) |
| D | A, T/A, T | 41 | (5.4) | 17 | (3.8) |
| E | G, C/G, C | 9 | (1.2) | 0 | (0.0) |

| | | | | |
|---|---|---|---|---|
| n = 1201 | 758 | (100) | 443 | (100) |

The above TaqMan method is a method developed by Applied Biosytems, and uses allele-specific probes and region-specific PCR primers having respective SNPs. Then the probes hybridize to the amplification region of PCR, fluorescence generates as the quencher of probe deviates according to the PCR amplification. By measuring the fluorescence, it can be determined whether the allele-specific probe has hybridized or not. Probes for detecting each SNP, and primer sets are as follows:

For −2352 typing:

(SEQ ID NO:61)

Probe for detecting G: AGGTGAGATAAAGCAA

(SEQ ID NO:62)

Probe for detecting A: TGAAAGGTGAAATAAA

(SEQ ID NO:63)

Primer pair: Forward: AAGGCCATTCAAAAGGATACATAAAA

(SEQ ID NO:64)

Reverse: TCTGTTTTCACTTTTGTTTTGCTTTG
For −934 typing:

(SEQ ID NO:65)

Probe for detecting A: TCCCCAATGATTCAG

(SEQ ID NO:66)

Probe for detecting G: CCCCAGTGATTCAG

(SEQ ID NO:67)

Primer pair: Forward: TGTGAACTTTGAAAGACGTGTCTACA

(SEQ ID NO:68)

Reverse: CAAGTAGAGAAACGCGCATCAG
For −692 typing:

(SEQ ID NO:69)

Probe for detecting T: TTCGAGTAGCGGCTC

(SEQ ID NO:70)

Probe for detecting C: TCGAGCAGCGGCT

(SEQ ID NO:71)

Primer pair: Forward: CCGCTTCGCTCTCTTTGC

(SEQ ID NO:72)

Reverse: CCTCTGCTTCTTTGAGCTTGGA
For 3435 typing:

(SEQ ID NO:73)

Probe for detecting C: CTCACGATCTCTTC

(SEQ ID NO:74)

Probe for detecting T: CCTCACAATCTCTT

(SEQ ID NO:75)

Primer pair: Forward: AACAGCCGGGTGGTGTCA

(SEQ ID NO:76)

Reverse: ATGTATGTTGGCCTCCTTTGCT

### EXAMPLE 15

In order to examine the direct association between the haplotypes and MDR1 promoter activity, the 5′ regulatory region between −2604 and −570 of genomic DNA from volunteers carrying three naturally occurring haplotypes (haplotypes 1, 2 and 3) was cloned. Then, the fragments to the reporter gene were ligated in the pGL3 basic vector. Because of the low frequencies of the polymorphisms at −1717 and −1325, genomic DNA with T monomorphic at −1717 and with A monomorphic at −1325 were used for reporter plasmid construction. These three constructs were then subjected to transient transfection in a human hepatoma cell line, HepG2. The promoter activity was analyzed after 48 h of transfection and normalized with the co-transfected phRL-TK activity. The relative luciferase activity is shown by a rate when the activity of haplotype 1 construct is set to 100%. Data are shown as mean value±S.D. (Standard Deviation) of the associated expression for each of the 4 individual experiments. Each experiment was estimated by using 3 dishes (P<0.05). As shown in Table 4, the minor-type construct carrying haplotypes 2 and 3 showed expression of 85.3±4.65% and 87.1±1.64%, respectively, of the major-type construct carrying haplotype 1. Together these experiments suggest that polymorphisms at the 5′ regulatory region affect the basal promoter activity of reporter constructs containing the human MDR1 gene upstream promoter region.

TABLE 5
Basal promoter activity of reporter constructs containing −2604 to
−570 of the human MDR1 gene harboring each haplotype

| | | | | | | Luciferase |
|---|---|---|---|---|---|---|
| haplotype | −2410 | −2352 | −1910 | −934 | −692 | activity (%) |
| 1 | T | G | T | A | T | 100 |
| 2 | T | A | T | A | T | 85.3 ± 4.65* |
| 3 | C | G | C | G | C | 87.1 ± 1.64* |

Relative luciferase activities are given as percentages of the activity of the haplotype 1 construct, which was considered 100%. The data are expressed as means ± S.D. of relative expression in four independent experiments. Each experiment was assayed using triplicate dishes.
*P <0.05

Relative luciferase activities are given as percentages of the activity of the haplotype1 construct, which was considered 100%. The data are expressed as means±S.D. of relative expression in four independent experiments. Each experiment was assayed using triplicate dishes. *P<0.05

### EXAMPLE 16

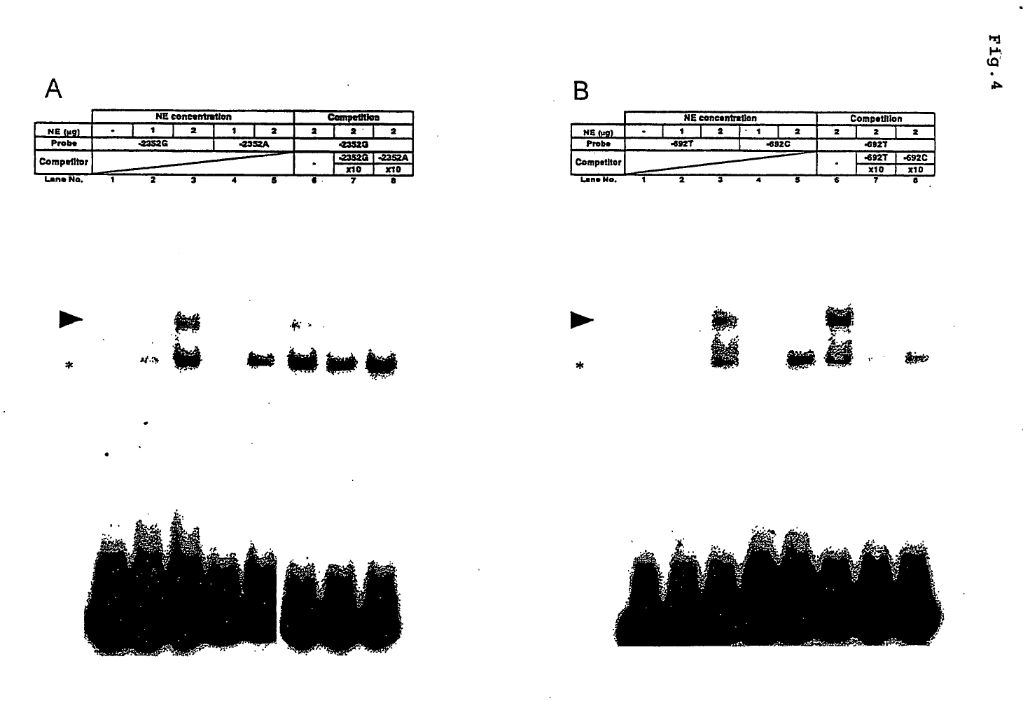

Electrophoretic mobility shift assays were used to investigate whether or not the SNPs of MDR15′ regulatory region altered binding of nuclear proteins. We first examined −2352G>A (FIG. 4A). A retarded band was observed when the probe −2352G was incubated with nuclear extracts of liver cells. This band was three times weaker than when −2352A was incubated. The specificity of the DNA-protein interaction was demonstrated by appropriate competition assays, i.e., the upper band almost completely disappeared under a 10-fold excess of the unlabeled oligonucleotide −2352G, while the addition of excess amounts of minor-type oligonucleotide −2352A did not inhibit the protein from binding to probe −2352G Then, −692T>C was examined (FIG. 4B). The allele-specific appearance of retarded band was also observed when the probe −692T was incubated with nuclear extracts. In competition assays, the upper band almost completely disappeared under a 10-fold excess of the unlabeled oligonucleotide −692T, and much weaker (nine times compared to −692T) inhibition of the binding was observed with the competitor −692C. Other oligonucleotide probes (−2410T>C, −1910T>C and −934A>G) showed no difference of protein-binding property between the major and minor types under the present conditions.

### EXAMPLE 17

Similarly to MDR1 gene, SNPs for human MRP2 gene encoding ABC transporter were examined. Genomic DNA was extracted from bone marrow comprising leukocytes collected from infant leukemia patients with their informed consent, and polymorphism were detected by direct sequencing method for all of 32 exons of MRP2 gene and 4 kb-upstream of promoter region, to identify 21 SNPs. The results are shown in Table 5. As for MDR 1 gene, probes for detecting each SNP, and primer sets are as follows:

For −3925 typing:

(SEQ ID NO:77)

Probe for detecting G: CTGGTTGTAGGGCTTT

(SEQ ID NO:78)

Probe for detecting A: CCTGGTTATAGGGCTTT

(SEQ ID NO:79)

Primer pair: Forward: CGGGCTTCATTCAGAATTTTTTATCTTT
GATT

(SEQ ID NO:80)

Reverse: CACCAAGTAGAACAAATGCCAAACA
For 3972 typing:

(SEQ ID NO:81)

Probe for detecting C: ATGCTACCGATGTCAC

(SEQ ID NO:82)

Probe for detecting T: ATGCTACCAATGTCAC

(SEQ ID NO:83)

Primer pair: Forward: TGGTCCTCAGAGGGATCACTT

(SEQ ID NO:84)

Reverse: TCCTTCACTCCACCTACCTTCTC
For −3933 typing:

(SEQ ID NO:85)

Probe for detecting C: AAGTAAGGTCTCTTTCC

(SEQ ID NO:86)

Probe for detecting T: AAGTAAGGTCTTTTTCC

(SEQ ID NO:87)

Primer pair: Forward: GCTTGCTGAGGAAAAGTTGGACATA

(SEQ ID NO:88)

Reverse: AGTTGCAGGAAATCAAAGATAAAAAATTCTGAA
For 2366 typing:

(SEQ ID NO:89)

Probe for detecting C: CATCCACTGCAGACAG

(SEQ ID NO:90)

Probe for detecting T: TCCACTGCAAACAG

(SEQ ID NO:91)

Primer pair: Forward: GCCAGAGCTACCTACCAAAATTTAGA

(SEQ ID NO:92)

Reverse: GCCTTTCAACAGGCCATTGG
For −924 typing:

(SEQ ID NO:93)

Probe for detecting G: AGGCCAAGGCAGAAG

(SEQ ID NO:94)

Probe for detecting A: AGGCCAAGACAGAAG

(SEQ ID NO:95)

Primer pair: Forward: GCAATCCCAGCCCTTTGG

(SEQ ID NO:96)

Reverse: CTCAAACTCCAGGCTTCAACAATC
For 1249 typing:

(SEQ ID NO:97)

Probe for detecting G: CTGTTTCTCCAACGGTGTA

(SEQ ID NO:98)

Probe for detecting A: ACTGTTTCTCCAATGGTGTA

(SEQ ID NO:99)

Primer pair: Forward: CCAACTTGGCCAGGAAGGA

(SEQ ID NO:100)

Reverse: GGCATCCACAGACATCAGGTT
For typing intron 19:

(SEQ ID NO:101)

Probe for detecting C: TCAAAGGAGAAGTGGTTTA

(SEQ ID NO:102)

Probe for detecting T: TTCAAAGGAGAAATGGTTTA

(SEQ ID NO:103)

Primer pair: Forward: CTGGATCTGAGTTTCTGGATTCTGT

(SEQ ID NO:104)

Reverse: GGGATGTTTTGCAAACTGTTCTTTG

TABLE 6
MRP2 genetic polymorphism of genes

| | Nucleic acid | Amino acid | Genotype | | | Allele frequency | |
|---|---|---|---|---|---|---|---|
| Location | Substitution | Substitution | Maj/Maj | Maj/Min | Min/Min | Major | Minor |
| exon 10 | G1249A | Val417IIe | 14 | 2 | 0 | 0.94 | 0.06 |
| exon 18 | C2366T | Ser789Phe | 15 | 1 | 0 | 0.97 | 0.03 |
| exon 22 | G2934A | Ser978Ser | 15 | 1 | 0 | 0.97 | 0.03 |
| exon 28 | C3972T | IIe1324IIe | 11 | 5 | 0 | 0.84 | 0.16 |
| 5′ flanking | C-24T | — | 11 | 5 | 0 | 0.84 | 0.16 |
| promoter | A-920G | — | 11 | 5 | 0 | 0.84 | 0.16 |
| promoter | G-924A | — | 2 | 12 | 2 | 0.50 | 0.50 |
| promoter | G-1450A | — | 11 | 5 | 0 | 0.84 | 0.16 |
| promoter | G-1675T | — | 3 | 11 | 2 | 0.53 | 0.47 |
| promoter | A-1847C | — | 15 | 1 | 0 | 0.97 | 0.03 |
| promoter | C-3133G | — | 11 | 5 | 0 | 0.84 | 0.16 |
| promoter | A-3414T | — | 9 | 6 | 0 | 0.80 | 0.20 |
| promoter | A-3459C | — | 10 | 5 | 0 | 0.83 | 0.17 |
| promoter | G-3925A | — | 3 | 1 | 0 | 0.53 | 0.47 |
| promoter | T-3993C | — | 10 | 6 | 0 | 0.81 | 0.19 |
| Intron 6 | T632 + 86A | — | 12 | 2 | 1 | 0.88 | 0.13 |
| Intron 14 | G1901-61A | — | 15 | 1 | 0 | 0.97 | 0.03 |
| Intron 19 | C2620-2133T | — | 8 | 7 | 1 | 0.72 | 0.28 |
| Intron 23 | C3258-56T | — | 11 | 5 | 0 | 0.84 | 0.16 |
| Intron 29 | G4146-584A | — | 14 | 1 | 1 | 0.91 | 0.09 |
| intron | C5199 + 317T | — | 11 | 5 | 0 | 0.84 | 0.16 |

According to the present invention, it is possible to determine haplotypes or diplotypes of MDR1 gene targeting the 5′ regulatory region of MDR1 gene, being expressed in the apical membrane side and being an ABC transporter transporting a wide range of substrates, and by using the determination results of haplotypes or diplotypes of the 5′regulatory region of MDR1 gene of each individual as a marker of drug responsiveness, as well as the fundamental knowledge concerning SNPs of the 5′regulatory region of MDR1 gene, it is possible to perform tailor made treatment. Furthermore, as it is useful as a marker for estimating an oncogenic risk and its development, it is possible to develop it to a tailor made prevention by estimating the risk of cancer.

The invention is further described by the following numbered paragraphs:

1. A method for determining haplotypes and/or diplotypes of a 5′regulatory region of MDR1 gene, by detecting a polymorphism at a position selected from −2903, −2410, −2352, −1910, −1717 and −1325, when the position is indicated in relation to a first base of translation start codon (ATG) which is set to +1, in a nucleotide sequence of a 5′regulatory region of a MDR1 gene.

2. A method for determining haplotypes and/or diplotypes of a 5′regulatory region of MDR1 gene, by detecting polymorphism at a position selected from −934 and/or −692 position, in addition to the polymorphism at a position selected from −2903, −2410, −2352, −1910, −1717 and −1325, when the position is indicated in relation to a first base of translation start codon (ATG) which is set to +1, in a nucleotide sequence of a 5′regulatory region of a MDR1 gene.

3. The method for determining haplotypes and/or diplotypes of a 5′regulatory region of MDR1 gene according to paragraph 1 or 2, comprising the step of investigating whether the base at −2903 is thymine or cytosine.

4. The method for determining haplotypes and/or diplotypes of a 5′regulatory region of MDR1 gene according to any one of paragraphs 1 to 3, comprising the step of investigating whether the base at −2410 is thymine or cytosine.

5. The method for determining haplotypes and/or diplotypes of a 5′regulatory region of MDR1 gene according to any one of paragraphs 1 to 4, comprising the step of investigating whether the base at −2352 is guanine or adenine.

6. The method for determining haplotypes and/or diplotypes of a 5′regulatory region of MDR1 gene according to any one of paragraphs 1 to 5, comprising the step of investigating whether the base at −1910 is thymine or cytosine.

7. The method for determining haplotypes and/or diplotypes of a 5′regulatory region of MDR1 gene according to any one of paragraphs 1 to 6, comprising the step of investigating whether the base at −1717 is thymine or cytosine.

8. The method for determining haplotypes and/or diplotypes of a 5′regulatory region of MDR1 gene according to any one of paragraphs 1 to 7, comprising the step of investigating whether the base at −1325 is guanine or adenine.

9. A DNA of 5′ regulatory region of MDR1 gene, wherein bases at −2410, −2352, −1910, −934, −692 are replaced with thymine, adenine, thymine, adenine, thymine, respectively, when the position is indicated in relation to a first base of translation start codon (ATG) which is set to +1, in a nucleotide sequence of a 5′regulatory region of MDR1 gene.

10. A DNA of 5′ regulatory region of MDR1 gene, wherein bases at −2410, −2352, −1910, −934, −692 are replaced with cytosine, guanine, cytosine, guanine, cytosine, respectively, when the position is indicated in relation to a first base of translation start codon (ATG) which is set to +1, in a nucleotide sequence of a 5′regulatory region of MDR1 gene.

11. A DNA of 5′ regulatory region of MDR1 gene, wherein bases at −2410, −2352, −1910, −934, −692 are replaced with cytosine, adenine, cytosine, guanine, cytosine, respectively, when the position is indicated in relation to a first base of translation start codon (ATG) which is set to +1, in a nucleotide sequence of a 5′regulatory region of MDR1 gene.

12. A DNA of 5′ regulatory region of MDR1 gene, wherein bases at −2410, −2352, −1910, −934, −692 are replaced with thymine, adenine, thymine, guanine, thymine, respectively, when the position is indicated in relation to a first base of translation start codon (ATG) which is set to +1, in a nucleotide sequence of a 5′regulatory region of MDR1 gene.

13. A primer set comprising a forward primer that hybridizes with a region upstream of a position for detecting polymorphism, and a reverse primer that hybridizes with a region downstream of a position for detecting polymorphism, which is used for a method for determining haplotypes of a 5′ regulatory region of MDR1 gene for detecting a polymorphism at a position selected from −2903, −2410, −2352, −1910, −1717 and −1325, when the position is indicated in relation to a first base of translation start codon (ATG) which is set to +1, in a nucleotide sequence of a 5′regulatory region of MDR1 gene.

14. A method for determining diplotype of 5′ regulatory region of MDR1 gene by detecting a polymorphism at −2352, and at a position selected from −2410, −1910 and −692, when the position is indicated in relation to a first base of translation start codon (ATG) which is set to +1, in a nucleotide sequence of a 5′regulatory region of MDR1 gene.

15. The method for determining diplotype of 5′ regulatory region of MDR 1 gene according to paragraph 14, wherein gene-typing is performed by TaqMan® method.

16. A probe and a primer set, used for a method for determining diplotype of 5′ regulatory region of MDR1 gene by detecting a polymorphism at −2352, and at a position selected from −2410, −1910 and −692, when the position is indicated in relation to a first base of translation start codon (ATG) which is set to +1, in a nucleotide sequence of a 5′regulatory region of MDR1 gene.

17. The probe and the primer set according to claim 16, used for a method for determining diplotype of 5′ regulatory region of MDR1 gene by TaqMan® method.

18. A method for estimating an onset of colon cancer, wherein the method for determining haplotypes and/or diplotypes of 5′ regulatory region of MDR1 gene according to any one of paragraphs 1 to 8, or the method for determining diplotype of 5′regulatory region of MDR1 gene according to claim 14 or 15 is used.

19. A method for developing a drug for controlling MDR1 expression, wherein at least one position selected from −2903, −2410, −2352, −1910, −1717 and −1325, −934, −692 is targeted, when the position is indicated in relation to a first base of translation start codon (ATG) which is set to +1, in a nucleotide sequence of a 5′regulatory region of MDR1 gene.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method for determining haplotypes and/or diplotypes of a 5′regulatory region of MDR1 gene, by detecting a polymorphism at a position selected from −2903, −2410, −2352, −1910, −1717 and −1325, and optionally by detecting polymorphism at a position selected from −934 and/or −692 position, when the position is indicated in relation to a first base of translation start codon (ATG) which is set to +1, in a nucleotide sequence of a 5′regulatory region of a MDR1 gene.

2. The method for determining haplotypes and/or diplotypes of a 5′regulatory region of MDR1 gene according to claim 1, comprising the step of investigating whether the base at −2903 is thymine or cytosine.

3. The method for determining haplotypes and/or diplotypes of a 5′regulatory region of MDR1 gene according to claim 1, comprising the step of investigating whether the base at −2410 is thymine or cytosine.

4. The method for determining haplotypes and/or diplotypes of a 5′regulatory region of MDR1 gene according to claim 1, comprising the step of investigating whether the base at −2352 is guanine or adenine.

5. The method for determining haplotypes and/or diplotypes of a 5′regulatory region of MDR1 gene according to claim 1, comprising the step of investigating whether the base at −1910 is thymine or cytosine.

6. The method for determining haplotypes and/or diplotypes of a 5′regulatory region of MDR1 gene according to claim 1, comprising the step of investigating whether the base at −1717 is thymine or cytosine.

7. The method for determining haplotypes and/or diplotypes of a 5′regulatory region of MDR1 gene according to claim 1, comprising the step of investigating whether the base at −1325 is guanine or adenine.

8. A DNA of 5′ regulatory region of MDR1 gene, wherein bases at −2410, −2352, −1910, −934, −692 are replaced with thymine, adenine, thymine, adenine, thymine, respectively, when the position is indicated in relation to a first base of translation start codon (ATG) which is set to +1, in a nucleotide sequence of a 5′regulatory region of MDR1 gene, or a DNA of 5′regulatory region of MDR1 gene, wherein bases at −2410, −2352, −1910, −934, −692 are replaced with cytosine, guanine, cytosine, guanine, cytosine, respectively, when the position is indicated in relation to a first base of translation start codon (ATG) which is set to +1, in a nucleotide sequence of a 5′regulatory region of MDR1 gene, or a DNA of 5′ regulatory region of MDR1 gene, wherein bases at −2410, −2352, −1910, −934, −692 are replaced with cytosine, adenine, cytosine, guanine, cytosine, respectively, when the position is indicated in relation to a first base of translation start codon (ATG) which is set to +1, in a nucleotide sequence of a 5′regulatory region of MDR1 gene, or a DNA of 5′ regulatory region of MDR1 gene, wherein bases at −2410, −2352, −1910, −934, −692 are replaced with thymine, adenine, thymine, guanine, thymine, respectively, when the position is indicated in relation to a first base of translation start codon (ATG) which is set to +1, in a nucleotide sequence of a 5′regulatory region of MDR1 gene.

9. A primer set comprising a forward primer that hybridizes with a region upstream of a position for detecting polymorphism, and a reverse primer that hybridizes with a region downstream of a position for detecting polymorphism, which is used for a method for determining haplotypes of a 5′ regulatory region of MDR1 gene according to claim 1.

10. A method for estimating an onset of colon cancer, wherein the method for determining haplotypes and/or diplotypes of 5′ regulatory region of MDR1 gene according to claim 1.

11. A method for developing a drug for controlling MDR1 expression, wherein at least one position selected from −2903, −2410, −2352, −1910, −1717 and −1325, −934, −692 is targeted, when the position is indicated in relation to a first base of translation start codon (ATG) which is set to +1, in a nucleotide sequence of a 5′regulatory region of MDR1 gene.
